(19) United States
(12) Patent Application Publication
(10) Pub. No.: US 2014/0356318 A1
(43) Pub. Date: Dec. 4, 2014

(54) ADOPTIVE CELL THERAPY WITH SPECIFIC REGULATORY LYMPHOCYTES

(71) Applicant: Israel Barken, San Diego, CA (US)

(72) Inventor: ISRAEL BARKEN, SAN DIEGO, CA (US)

(21) Appl. No.: 13/904,010

(22) Filed: May 28, 2013

(51) Int. Cl. A61K 35/14; A61K 38/20; A61K 38/18; G01N 33/50

(52) U.S. Cl. CPC A61K 35/17; G01N 33/5094; A61K 38/2066; A61K 38/1841
USPC 424/85.2; 424/93.71; 435/34; 435/375

(57) ABSTRACT

The present invention comprises a method of treatment of an autoimmune disease involving a specific tolerance induction (“STI”) event, wherein the method includes: collecting a first sample from the patient prior to the STI event; detecting an STI event or performing a procedure that correlates in time to an STI event; collecting a second sample from the patient after the STI event; preparing lymphocytes from the first and second samples; preparing and sequencing DNA or cDNA from the prepared lymphocytes; identifying sequences of prevalent T or B cell receptors (“prevalent receptor sequences”) among the lymphocytes of the second sample; selecting a regulatory lymphocyte that carries at least one prevalent receptor sequence, which selected regulatory lymphocyte (i) expresses at least one prevalent receptor sequence or (ii) is generated from an autologous or allogeneic naïve lymphocyte, which naïve lymphocyte is engineered and induced to become a regulatory lymphocyte that expresses at least one prevalent receptor sequence; culturing the selected regulatory lymphocyte, thereby generating daughter cells of said regulatory lymphocyte; and administering the daughter cells to the patient.

### CROSS REFERENCE TO RELATED APPLICATIONS

This application claims priority to U.S. Ser. No. 13/598,611 and U.S. Ser. No. 13/598,603, both of which were filed Aug. 29, 2011, and both of which claim priority to U.S. Ser. No. 61/528,657, filed Aug. 29, 2011; all of the identified applications are hereby incorporated in their respective entireties herein.

### BACKGROUND OF THE INVENTION

The field of the present invention relates generally to the field of medical immunology and, more specifically, to autoimmune diseases in humans and animals. In particular, the present invention relates to the identification and use of regulatory immune cells that arise in a patient for the design and generation of a specific cell-based immunological treatment to prevent, ameliorate, or cure autoimmune diseases.

Autoimmune diseases in humans and animals start by the ordinary workings and errors of an individual's own immune system. The immune system is designed to generate B and T cells to protect against foreign bodies; but the mechanisms necessary to create sufficient diversity in the receptors of these immune cells also generate B and T cells that attack self, i.e., autoreactive immune cells. Other mechanisms exist in the immune system to weed out such autoreactive immune cells. Disease in this arena occurs where and when the weeding machinery fails to down-regulate the autoreactive immune cells or cause them to die.

When responding to an event or treatment that highlights the focus of the autoimmune disease, the patient's immune system generally mounts a reaction where the repertoire of cells of the patient's lymphocyte population changes to counteract the autoreactive cells, but does so inadequately. In other words, the patient's immune system responds positively to treatment but does so insufficiently and, thus, the immunological attack on self persists.

The immune system's mission is to attack perceived new antigens, which process includes an upsurge of immune cells that target particular antigens or portions thereof that are associated with a foreign body, such as a virus or a bacterium. That aspect of the upsurge of immune cells is certainly advantageous. However, at the same time that advantageous immune cells are generated, it is also the case that deleterious autoreactive immune cells are also generated, as noted above. Part of the immune system's normal functioning is also to produce regulatory lymphocytes to down-regulate or cause the death of the autoreactive immune cells, which, in the context of one variety of such regulatory lymphocytes, can be accomplished by raising the concentration of regulatory T cells (“T_{reg} cells”) that have T cell receptors (“TCRs”) that mediate the down-regulation. Accordingly, if a patient's immune system generates a sufficient concentration of T_{reg} cells having TCRs specific for action against the specific autoreactive T cells that are responsible for the damage associated with the autoimmune disease, then the patient will not experience damage from the autoimmune disease. However, if the patient's immune system generates an insufficient concentration of T_{reg} cells having TCRs specific for action against the specific autoreactive T cells that are responsible for the damage associated with the autoimmune disease, then the patient will experience damage from the autoimmune disease despite having produced some perfectly helpful T_{reg} cells.

A subset of T_{reg} cells is referred to as a “clonotype”; each cell of a T_{reg} clonotype includes an identical TCR that allows the T_{reg} cells of the clonotype to down-regulate the effector cells attacking a particular epitope, i.e., an immunological target associated with (i) a foreign body, for down-regulating the desired immune response once invasion by the virus or bacteria, for example, has been overcome, or (ii) a cell or tissue of the patient, for down-regulating the undesired immune response. Effector cells targeting self are referred to herein as “autoreactive cells.” Each clonotype of autoreactive cells mounts an attack on a particular antigen that is perceived as foreign; in contrast, each T_{reg} cell clonotype regulates the activity of specific autoreactive cells.

Efforts have been made to identify sets of T_{reg} cells that can serve to down-regulate an immunological attack on self tissue. Indeed, such T_{reg} cells have been studied and shown to be effective in murine models of diabetes mellitus type I and systemic lupus erythematosus. Riley et al., IMMUNITY 30:657-665 (2009). There is a current clinical study (started in 2010; estimated completion in December 2016) in which 14 diabetic individuals are undergoing treatment by isolation of their respective T_{reg} populations using beads coated with anti-CD3 and anti-CD28 (to which the class of T_{reg} cells will adhere under appropriate conditions known in the art), then expanding their respective total T_{reg} populations by incubation in media containing interleukin-2 (“IL-2”), and, finally, infusing the expanded total populations of T_{reg} cells back into the respective patients. See UCSF T1DM Immunotherapy Study, ClinicalTrials.gov Identifier No. NCT01210664, verified November 2012, viewable at http://www.clinicaltrials.gov/ct2/show/NCT01210664?term=T+cell+autoimmune+therapy&rank=7.

Unfortunately, treatments in use today that have been demonstrated to lessen the severity of autoimmune disease symptoms are not uniform in their effect across patients or with respect to a single patient in treating successive flare-ups of autoimmune symptoms. Moreover, the current treatments are not specific to the immune system cells that cause the ill-effects but, instead, down-regulate or otherwise minimize the effect of the patient's immune system generally.

Methods do not exist today that, irrespective of mechanism, are reliably effective time after time against a flare up of an autoimmune disease. Nor is there available today a method to present signals for down-regulating one's immune system with specificity to the particular group of autoreactive cells that are causing the ill-effect. Indeed, while it is desired that the clinical study regarding diabetes identified above will bear positive results, its overall design precludes specificity in view of its reliance on the total population of CD4⁺/CD28⁺ T_{reg} cells present in each patient's blood. In other words, that study's protocol may indeed down-regulate the immune activity at pancreatic islets sites that is the direct cause of the disease, but, in so doing, other sites that benefit from the patient's immune activity will also be down-regulated by that course of treatment.

Therefore, there is a need for improved methods for treating autoimmune diseases. Learning and applying approaches built into humans and animals that may not reach a clinically detected result is a path of treatment that is currently unavailable. The present invention presents novel methods of analysis and treatment that provides protocols for building efficacy into a quantity of an individual's complement of lymphocytes that are geared to regulate the attack on self tissue or organs that are mistakenly perceived as foreign objects by the patient's own immune system.

### SUMMARY OF THE INVENTION

In its various embodiments, the inventive method of treatment of a patient who suffers from an autoimmune disease, wherein the patient undergoes a specific tolerance induction (“(“STI”) event, can include variations of the following steps: optionally collecting a first lymphocyte-containing sample from the patient prior to the STI event (“pre-STI event sample”); detecting an STI event or performing a procedure that correlates in time to an STI event; drawing a second lymphocyte-containing sample from the patient, preferably from the same source as the first lymphocyte-containing sample, after the STI event (“post-STI event sample”); preparing lymphocytes from the pre-STI event sample or the post-STI event sample; preparing and sequencing DNA or cDNA derived from the prepared lymphocytes; identifying sequences of prevalent T cell receptors (“TCRs”) or B cell receptors (“BCRs”) (collectively, “prevalent receptor sequences”) derived from the post-STI event sample; selecting a regulatory lymphocyte that carries at least one of the prevalent receptor sequences or a sequence that is about 85% identical or greater with respect to one of the prevalent receptor sequences, which selected regulatory lymphocyte (i) expresses one or more of the prevalent receptor sequences or (ii) is generated from an autologous or allogeneic naïve lymphocyte, which naïve lymphocyte is engineered and induced to become a regulatory lymphocyte that expresses a receptor sequence that is about 85% identical or greater, or about 90% identical or greater, or about 95% identical or greater, or about 98% identical or greater with respect to one of the prevalent receptor sequences; culturing the selected regulatory lymphocyte, thereby generating daughter cells of said regulatory lymphocyte; and administering said daughter cells to said patient.

The inventive method can be employed irrespective whether a pre-STI event sample is collected or not; but if indeed, for example, a lymphocyte-containing sample was collected from the patient prior to the STI event, then the step of identifying DNA or cDNA sequences of prevalent TCRs or BCRs among the DNA or cDNA of the lymphocytes of the post-STI event sample further comprises identifying prevalent TCRs or BCRs from the post-STI event sample relative to TCRs or BCRs, respectively, from the pre-STI event sample. Alternative lymphocyte-containing samples that are usefully employed include blood, lymph, one or more lymph nodes, marrow, spinal fluid (if a site of inflammation as associated with a spinal cord or brain injury, for example), site of autoimmune attack, and inflamed tissue.

Accordingly, the inventive method can, in one embodiment, further comprise the step of selecting clonotypes from the post-STI event sample that are not identified among clonotypes from the pre-STI event sample or that have an expansion frequency of about 0.5% or greater relative to the clonotypes from the pre-STI event sample. Part of this embodiment includes the further step of selecting clonotypes from the post-STI event sample that are not identified among clonotypes from the pre-STI event sample or that have an expansion frequency of 0.5% or greater relative to the clonotypes from the pre-STI event sample.

These and additional embodiments of the present invention are further described and enabled by the specification and figures that follow.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings, of which:

FIG. 1 is a flowchart corresponding to numerous protocols for the in vitro and in vivo study, generation, and use of selected regulatory T or B cells in the context of the present invention.

FIG. 2 is a more particularized flowchart corresponding to one embodiment of the present invention.

FIG. 3 presents excerpts from a sample report provided by a TCR profiling vendor (Evrotec).

FIG. 4 presents further excerpts from a sample report provided by a TCR profiling vendor (Evrotec).

FIG. 5 is a scatterplot demonstrating the reproducibility of the receptor profiling method, provided by a TCR and BCR profiling vendor (Adaptive Biotechnologies Corporation, Seattle, Wash.).

### DETAILED DESCRIPTION OF THE INVENTION

The present invention provides a method of treatment of a patient who suffers from an autoimmune disease that causes an immunological attack on self, i.e., on the patient's own tissue or cells. The present invention includes a product for treating a patient afflicted with an autoimmune disease usefully employed in the context of the inventive method. The product and the method of treatment harnesses the strategy of a patient's own immune system to create a new cellular product usefully employed in the context of the inventive method disclosed herein to treat more efficiently the patient's autoimmune disease.

Autoimmune disease can attack any tissue or organ of the body; thus far over 80 such diseases have been described in the medical literature, and there is no doubt that more autoimmune diseases remain to be described. Some autoimmune diseases are systemic and attack at multiple sites, such as systemic lupus erythematosus or rheumatoid arthritis; other autoimmune diseases are highly specific in attacking specific cell types or organs, such as type 1 diabetes or celiac disease. It is striking but nonetheless the case that the present invention is amenable to treatment of any autoimmune disease or injury made worse by the patient's own autoreactive immune activity, such as occurs in a brain or heart infarct or a brain or spinal cord injury.

Accordingly, the set of diseases amenable to treatment by the inventive method and materials include all disease or injury conditions that are caused or exacerbated by immune activity against self, which disease or injury conditions include, but are not limited to, Addison's disease, alopecia, Alzheimer's disease, amyotrophic lateral sclerosis (“ALS”), aplastic anemia, autoimmune brain infarction, autoimmune coronary artery disease, autoimmune myocardial infarction, autoimmune periodontal disease, brain trauma, celiac disease, Crohn's disease, diabetes mellitus type I, glaucoma, glutamate toxicity, idiopathic thrombocytopenic purpura, interstitial cystitis, multiple sclerosis, Parkinson's disease, psoriasis, rheumatoid arthritis, brain or spinal cord trauma, systemic lupus erythematosus, and systemic sclerosis, and the like. Preferably, the present method is applied to Alzheimer's disease, aplastic anemia, Crohn's disease, diabetes mellitus type I, idiopathic thrombocytopenic purpura, Parkinson's disease, psoriasis, rheumatoid arthritis, and spinal cord trauma.

These and other applications of the inventive methods are amply set forth herein. As indicated, any injury or autoimmune disease can be cured or ameliorated as to its symptoms by observing what the policing immune system is trying to do, and then helping the body's internal defenders mount an efficacious treatment along the same lines. While it is clearly contemplated that the present invention is amenable for augmenting the immune system reaction that occurs in the context of any disease or injury where harm is exasperated by immunological attack on self tissue, it is worthwhile highlighting a few of these. Parkinson's Disease, associated with degeneration of dopamine neurons in the substantia nigra pars compacta part of the brain where the role of inflammation mediates the innate and adaptive immune systems. Panaro and Cianciulli, CURR. PHARM. DES. 18(2):200-8 (2012). The present inventive method is also amenable for addressing interstitial cystitis and periodontal disease. With regard to periodontal disease, there is little doubt of an STI event occurring with each visit to the dentist's office, particularly if one's teeth are cleaned during the visit. A blood draw before and after such a visit reveals new TCR and BCR abundancies rising in the patient's receptor repertoires, and thus forms the basis for building an immunotherapy for increasing the regulatory lymphocytes to address the inflammatory processes acting on that patient's gum tissue. The discussion that follows will further highlight various of these autoimmune diseases and traumas, showing protocols for harnessing and augmenting underlying immune system processes that are indeed common and readily implementable by those skilled in the immunotherapeutic arts.

For the limited purpose only of increasing the clarity of how to use and how to make the present invention, applicant's working hypothesis of the underlying mechanism behind the inventive methods and materials is presented herein below. There being no requirement to disclose the mechanism by which an invention works, nor the inventor's working hypothesis, applicant is not bound by any theory or hypothesis presented here. However, applicant presents his working hypothesis as doing so helps lay a framework for the reader to comprehend the materials and methods disclosed here. Succinctly and particularly, the working hypothesis is that a treatment for an autoimmune disease that has been shown to ameliorate symptoms in one patient is likely to repeat even if the same patient or a second patient does not later experience the same or similar clinical response to the same treatment. Furthermore, the same treatment causes events geared to the positive outcome even when no positive clinical result is observed. Accordingly, the treated patient mounts an immunological attack to reverse symptoms of the autoimmune disease irrespective whether a positive clinical result obtains or not.

This working hypothesis provides that the treatment that has been shown to be effective in ameliorating or reversing ill-effects of an autoimmune disease induces generation of immune system regulatory cells that, when wholly successful, mediate specific tolerance with regard to the autoreactive cells that are causing grief to the patient.—But the actors that mediate the specific tolerance may not be present in sufficient numbers to provide a positive clinical effect; or receptors of the so-induced regulatory cells may not sufficiently recognize the targeted autoreactive cells. If a patient's immunological responses to the treatment are studied at the sub-cellular level, then one can identify which are the regulatory cells that are arising in response to that treatment and suitable regulatory cells isolated, created, generated—for provisioning of a composition for more effective treatment. Such a sub-cellular study has been rendered doable by developments in DNA sequencing technologies developed over the past decade or so, including high throughput sequencing. See Soon et al., MOLECULAR SYSTEMS BIOLOGY 9:640 (2013). The disclosure provided herein below details a variety of embodiments of a new method that is predicated on the availability of sequencing power as noted and applying such sequence-based information to create custom compositions for adoptive cell therapy designed for a patient.

The phenomenon of providing a particular treatment for an autoimmune disease that results in an immunological response that ameliorates or controls clinical symptoms of the disease at least historically, if not in the immediate instance, is referred to herein as a specific tolerance induction (“STI”) event. For example, application of certain photodynamic therapy (referred to as “PUVA”; more particularly discussed below) has been shown to result in an immunological response associated with good effect for psoriasis patients (psoriasis is an autoimmune disease of the epidermis). But the photodynamic therapy does not uniformly result in relief; nonetheless, application of the photodynamic therapy results in an immunological response. Accordingly, with respect to this example, the photodynamic therapy is or correlates in time to an STI event. By analogy, a treatment of an autoimmune disease that has resulted in an immunological response that ameliorates or controls symptoms of the disease is or correlates in time to an STI event.

STI events occur not only in response to induced scenarios due to a medical treatment, but also occur in response or correlation to natural but deleterious scenarios, such as a flare (i.e., marked increase in symptoms) of an existing autoimmune disease or an infarct of heart or brain tissue; and accidental scenarios, such as received trauma resulting in a brain or spinal cord injury. In each such scenario, the patient's immune system begins mounting a beneficial immune response of raising its concentration of regulatory lymphatic cells dedicated to down-regulating the built-in immune attack on new antigens arising from the damaged tissue (which are actually native antigens perceived as new). The immune-based clearance of damaged tissue is, of course, an appropriate and necessary function; however, that response must be controlled to protect healthy tissue as well as to protect damaged tissue that may heal. Accordingly, increasing the down-regulatory paths can lessen the harm done by the heart attack or stroke patient's own immune system.

The immune-based clearance can include otherwise perfectly normal tissue in the instance of standard autoimmune disease, or injured or dead tissue in the instance of trauma or infarct.

### DEFINITIONS

All technical terms have the standard accepted meaning in the art to which the present disclosure applies unless otherwise defined herein. Certain definitions may be found in U.S. Pat. No. 5,830,755, which is incorporated herein for the purpose of supplying definitions. Definitions presented herein shall control should there be conflict in definitions between those presented here and “standard accepted meanings” or those of any incorporated U.S. patent publication recited here.

The term “adjuvant” as used herein refers to a pharmacological or immunological agent that modifies the effect of other agents, such as a drug or vaccine. They are often included in vaccines to enhance the recipient's immune response to a supplied antigen, while keeping the injected foreign material to a minimum. Adjuvants in immunology are often used to modify or augment the effects of a vaccine by stimulating the immune system to respond to the vaccine more vigorously, and thus providing increased immunity to a particular disease. Adjuvants accomplish this task by mimicking specific sets of evolutionarily conserved molecules, so called PAMPs, which include liposomes, lipopolysaccharide (LPS), molecular cages for antigen, components of bacterial cell walls, and endocytosed nucleic acids such as double-stranded RNA (dsRNA), single-stranded DNA (ssDNA), and unmethylated CpG dinucleotide-containing DNA. Because immune systems have evolved to recognize these specific antigenic moieties, the presence of an adjuvant in conjunction with the vaccine can greatly increase the innate immune response to the antigen by augmenting the activities of dendritic cells (DCs), lymphocytes, and macrophages by mimicking a natural infection. Furthermore, because adjuvants are attenuated beyond any function of virulence, they pose little or no independent threat to a host organism. One adjuvant usefully employed in the context of the present invention is BCG.

The term “adoptive cell therapy” as used herein refers to a cell-based immunotherapy that, as used herein, relates to the transfusion of autologous or allogenic lymphocytes, identified as T or B cells, genetically modified or not, that have been expanded ex vivo prior to said transfusion.

The term “autoimmune target tissue” as used herein refers to any tissue or structure attacked by one's immune system irrespective whether the attacked tissue or structure started off without disease or included cell rupture due to trauma or infarct.

The term “autoreactive cell” as used herein refers to a lymphocyte that attacks self antigens; they can be B cells, T cells, or other immune system cells that are commonly referred to as effector cells.

The term “Bacillus Calmette-Guerin” or (“BCG”) as used herein refers to an agent that promotes the immunological reaction of response to an autoimmune disease; BCG can comprise a variety of components that includes a strain of attenuated (i.e., weakened) live bovine tuberculosis bacillus, Mycobacterium bovis, that has lost its virulence in humans (with respect to human patients); may alternatively include Mycobacterium tuberculosis that has been rendered unable to cause tuberculosis in a human patient; and, analogously for non-human patients, such tuberculosis bacilli rendered or that are avirulent with respect to the particular non-human patient to be treated.

The term “B cell” as used herein refers to certain lymphocytes (which are also referred to as white blood cells) that are a vital part of the immune system—specifically the humoral immunity branch of the adaptive immune system. B cells can be distinguished from other lymphocytes, such as T cells and natural killer cells (NK cells), for example, by the presence of a protein on the B cell's outer surface known as a B cell receptor.

The term “B cell receptor” or (“BCR”) as used herein refers to a transmembrane receptor protein located, in part, on the outer surface of B cells. The receptor's binding moiety is composed of a membrane-bound antibody that, like all antibodies, has a unique and randomly determined antigen-binding site.

The term “biopsy” as used herein refers to a medical procedure involving the removal of cells or tissues for examination to determine the presence or extent of a disease. The tissue is generally examined optically under a microscope by a pathologist, and can also be analyzed chemically. When an entire lump or suspicious area is removed, the procedure is called an excisional biopsy. When only a sample of tissue is removed with preservation of the histological architecture of the tissue's cells, the procedure is called an incisional biopsy or core biopsy. When a sample of tissue or fluid is removed with a needle in such a way that cells are removed without preserving the histological architecture of the tissue cells, the procedure is called a needle aspiration biopsy.

The term “clonotype” as used herein refers to a set of cells that are substantially similar and/or identical (i.e., clonal). More specifically as used herein, a clonotype of lymphocytes refers to cells that each have the same or highly similar TCR or BCR.

The term “cluster of differentiation” (often abbreviated as CD) as used herein refers to a protocol used for the identification and investigation of cell surface molecules present on white blood cells, providing targets for immunophenotyping of cells. Physiologically, CD molecules can act in numerous ways, often acting as receptors or ligands (the molecule that activates a receptor) important to the cell. A signal cascade is usually initiated, altering the behavior of the cell (see cell signaling). Some CD proteins do not play a role in cell signaling, but have other functions, such as cell adhesion. CD for humans is numbered up to 350 most recently (as of 2009).

The term “CD8” (cluster of differentiation 8) as used herein refers to a transmembrane glycoprotein that serves as a co-receptor for the T cell receptor (TCR). Like the TCR, CD8 binds to a major histocompatibility complex (MHC) molecule, but is specific for the class I MHC protein. There are two isoforms of the protein, alpha and beta, each encoded by a different gene. In humans, both genes are located on chromosome 2 in position 2p12.

The term “copy DNA” or “cDNA” as used herein refers to single- or double-stranded DNA where one strand is complementary to a certain sequence of messenger RNA (“mRNA”). It is usually formed in a laboratory by the action of the enzyme reverse transcriptase that reverse transcribes the sequence of nucleotides of RNA into corresponding complementary DNA nucleotides. A “cDNA library” is a collection of such DNAs that reflects the repertoire of mRNAs found in cells harvested for this purpose.

The term “formalin-fixed, paraffin-embedded” or (“FFPE”) as used herein refers to a common way to preserve tissue samples well known to pathologists and microscopists.

The term “high throughput sequencing” or “HTS” as used herein refers to next-generation DNA sequencing that combines methods for amplifying specified portions of the genome, such as multiplex polymerase chain reaction (“mPCR”) methods (for amplifying specific genomic regions), with a method of sequencing multiple molecules en masse, in accordance, for example, with procedures set forth in Robins, et al., BLOOD 114:4099-4107 (2009); Boyd et al., U.S. Patent Application 20120220466 (2012); Faham and Willis, U.S. Patent Application 20120135409 (2012), which recited methods and discussions thereof are hereby incorporated herein by reference.

The term “identity” as a percentage as used herein with respect to two DNA or cDNA or protein molecules, respectively, refers to the degree of homology or similarity in the sequence of nucleotides or amino acids, as appropriate, between the compared molecules, which degree can be established using a mathematical algorithm as known in the art. A preferred, non-limiting example of a mathematical algorithm utilized for the comparison of two sequences is the algorithm of Karlin and Altschul, PROC. NAT'L ACAD. SCI. USA 87:2264-2268 (1990), modified as in Karlin and Altschul, PROC. NAT'L ACAD. SCI. USA 90:5873-5877 (1993). Such an algorithm is incorporated into the NBLAST and XBLAST programs of Altschul et al., J. MOL. BIOL. 215:403-410 (1990). BLAST nucleotide searches can be performed with the NBLAST program, score=100, wordlength=12 to obtain nucleotide sequences similar or homologous to nucleic acid molecules usefully employed in the context of the invention. To obtain gapped alignments for comparison purposes, Gapped BLAST can be utilized as described in Altschul et al., NUCLEIC ACIDS RES. 25:3389-3402 (1997). When utilizing BLAST and Gapped BLAST programs, the default parameters of the respective programs (e.g., XBLAST and NBLAST) can be used. See http://www.ncbi.nlm.nih.gov. The “percent identity” between two sequences can be determined using techniques similar to those described above, with or without allowing gaps.

The term “isolated” or “purified” with regard to a population of cells as used herein refers to a cell population that either has no naturally-occurring counterpart or has been substantially separated or purified from other components, including other cell types, which naturally accompany it, e.g., in normal or diseased tissues such as lung, kidney, or colon, or body fluids such as blood or serum. Typically, an isolated cell population is at least two-fold, four-fold, or eight-fold enriched for a specified cell type when compared to the natural source from which the population was obtained.

Further regarding the term “isolated” or “purified” with regard to a population of cells as used herein, a population or subpopulation of cells that is “substantially” of a specified cell type is one that has a count of the specified cell type that is about 50%, about 75%, about 80%, about 90%, about 95% or, most preferably, about 98% or about 99% of the total cell count of the population or subpopulation or one which is about two-fold, about four-fold, about eight-fold, about ten-fold or about 20-fold enriched for a specified cell type as compared to a source population of the specified cell type. In other embodiments, the isolated or purified population of cells with respect to the specified cell type is at least 50%, at least 75%, at least 80%, at least 90%, at least 95% or, most preferably, at least 98% or at least 99% of the total cell count of the population or subpopulation or one which is at least two-fold, at least four-fold, at least eight-fold, at least ten-fold or at least 20-fold enriched for a specified cell type as compared to a source population of the specified cell type. A substantially pure population may be at least about 50% regulatory lymphocytes in the population, at least about 75% regulatory lymphocytes in the population, at least about 80% regulatory lymphocytes in the population, at least about 90% regulatory lymphocytes in the population, at least about 95% regulatory lymphocytes in the population, at least about 98% regulatory lymphocytes in the population, or at least about 99% regulatory lymphocytes in the population.

The terms “patient,” “individual” and “subject,” as used herein, are fully synonymous inter se and can be used interchangeably, and do not necessarily refer to an animal or person who is ill or sick (i.e., the terms can reference a healthy individual or an individual who is not experiencing any symptoms of a disease or condition). The methods and compositions disclosed herein are suitable for use in or with regard to an individual that is a member of the Vertebrate class, Mammalia, including, without limitation, primates, livestock and domestic pets (e.g., a companion animal).

The term “regulatory lymphocytes” as used herein refers to any lymphocyte that exerts control or influence over other cells having immune function, which includes T cells, B cells, and antigen-presenting cells, such as dendritic cells.

The term “sample” or “biological sample” or “lymphocyte-containing sample” as used herein refers to tissues or body fluids removed from a mammal, preferably a human or companion animal, and which contain lymphocytes. Samples preferably are blood and blood fractions, including peripheral blood, lymph glands or fluid therefrom, or tissue that is subject to autoimmune attack. Methods for obtaining such samples are well known to workers in the fields of cellular immunology, pathology, and surgery. They include sampling blood in well known ways, or obtaining biopsies from the thymus or other tissue or organ.

The term “T cell” as used herein refers to certain white blood cells, also known as lymphocytes, that play a central role in cell-mediated immunity. They can be distinguished from other lymphocyte types, such as B cells and natural killer cells (NK cells) by the presence of a special receptor that emanates from their cell surface and is called a T cell receptor (“TCR”).

The term “T cell receptor” or “TCR” as used herein refers to a molecule found on the surface of T cells that is, in general, responsible for recognizing antigens bound to major histocompatibility complex (MHC) molecules. The binding between TCR and antigen is of relatively low affinity and is degenerate: that is, many TCRs recognize the same antigen and many antigens are recognized by the same TCR. The TCR is composed of two different protein chains (that is, it is a heterodimer). In 95% of T cells in peripheral blood, this consists of alpha (α) and beta (β) chains, whereas in 5% of T cells in peripheral blood, this consists of gamma and delta (γ/δ) chains. These percentages differ in other locations in the body.

The term “treatment” as used herein refers to clinical intervention in an attempt to alter the natural course of the individual or cell being treated, and may be performed either for prophylaxis and/or during the course of clinical pathology. Desirable effects include preventing occurrence or recurrence of disease, alleviation of symptoms, diminishment of any direct or indirect pathological consequences of the disease, lowering the rate of disease progression, amelioration or palliation of the disease state, and remission or improved prognosis. Accordingly, a therapeutic benefit is not necessarily a cure for a particular disease or condition, but rather, preferably encompasses a result which most typically includes alleviation of the disease or condition, elimination of the disease or condition, reduction of a symptom associated with the disease or condition, prevention or alleviation of a secondary disease or condition resulting from the occurrence of a primary disease or condition (e.g., glaucoma secondary to type 1 diabetes), and/or prevention of the disease or condition.

The term “VDJ recombination” or “V(D)J recombination” or “somatic recombination” as used herein refers to a mechanism of genetic recombination in the early stages of immunoglobulin (Ig) and T cell receptor (TCR) production. V(D)J recombination nearly-randomly combines Variable, Diverse, and Joining gene segments in vertebrates, and, because of its randomness in choosing different genes, is able to encode proteins to match diverse antigens from bacteria, viruses, parasites, dysfunctional cells such as tumor cells, and pollens. V(D)J combinations can be studied in accordance with, for example, the methods set forth in Pasqual and Weisbuch, U.S. Patent Application US20110003291 (2011), of which the methods and discussion are hereby incorporated by reference.

Summarizing the introductory discussion above, the inventive product used comprises regulatory lymphocytes that are selected based on how the patient reacts to a specific tolerance induction (“STI”) event arising from a flare up of symptoms or from the administration of a procedure designed to ameliorate or control the autoimmune disease. The selected lymphocytes are identified by inclusion of receptors that arise anew or increase substantially in concentration relative to the receptors found among all other lymphocytes of a lymphocyte-containing tissue, such as peripheral blood, a lymph gland, or immune-targeted tissue, as compared to receptors found in lymphocytes from same source of lymphocytes from prior to the specific tolerance induction event.

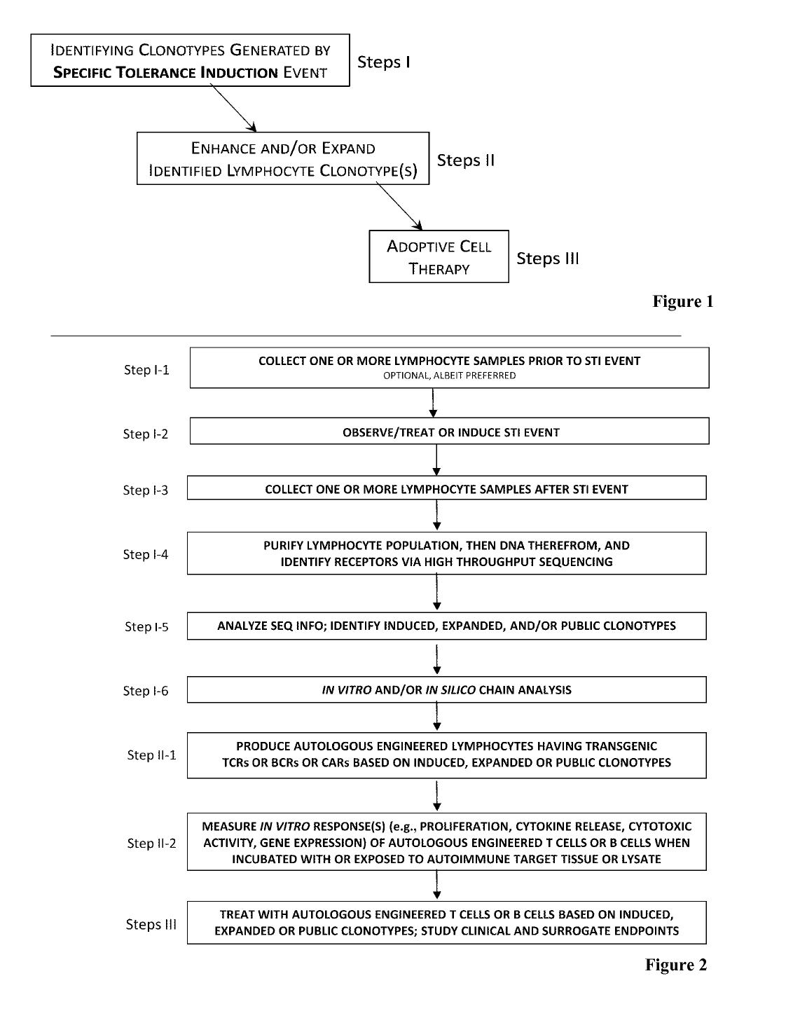

At its core, the present invention results from careful observation of a patient's native mechanisms that respond to the autoimmune disease or injury, but which native mechanisms fail. As outlined in FIG. 1, the present invention includes steps for:
(1) identifying clonotypes generated by an STI event, wherein protocols are followed that detect the body's own path to cure or ameliorate the ill-effects of autoreactivity of an autoimmune disease in response to the STI event,
(2) protocols for generating autologous or allogeneic cells that can more fully implement that path, which cells may be enhanced and/or expanded but unaltered, or both altered and expanded, and
(3) generation of a cocktail of such cells that are the implementers of the path upon infusion of the cocktail of the implementing cells, i.e., adoptive cell therapy.

Enhancement of the native mechanism in this context takes one of three forms: (1) Observing the rise of regulatory immune cells coincident in time, or substantially coincident, to an event that triggers an attempt toward immuno-tolerance, raising daughter cells of the observed regulatory immune cells and introducing them into the patient; (2) once identifying the rising regulatory immune cells, creating a genetically engineered autologous cell that includes receptors altered to improve affinity for likely targeted effector cells and/or increasing the concentration of such receptors on such cells; or (3) combining both strategies, i.e., presenting an increased population of daughters of the very cells found to rise in concentration upon tolerance induction as well as cells that are appropriately engineered to have increased affinity for the targeted effector cells.

Accordingly, in certain embodiments of the treatment detailed herein reintroduces selected, sometimes altered lymphocytes of the patient to the patient. In contrast to current protocols that reinfuse the full panoply of patient lymphocytes that include the full complement of receptors found on such cells at the time the cells were collected or, at most, reducing the diversity to a sub-population of all regulatory T cells or regulatory B cells, the present invention reinfuses a refined subpopulation of the lymphocytes that include no more than one up to a few different T cell receptors (TCRs) and/or B cell receptors (BCRs) and/or modified such receptors, such as chimeric antigen receptors (CARs).

This protocol results, at minimum, in ameliorating symptoms of an autoimmune disease; and, at best, prevents future disease or controls current disease—accomplished in a manner that reduces the risk of introducing increased concentrations of effector cells generally or of regulatory cells that if increased in concentration may down-regulate effector cells whose functions are beneficial.

Autoimmune disease symptoms include pain, reduction in mass and/or function of attacked tissue, reduction in flexibility (particularly with respect to affected joints or connective tissue), lesions on skin- or organ-directed disease, and the like. These biological factors are observed and provide both objective and subjective gauges for assessing the effectiveness of treatment using the methods and products disclosed here.

Returning to FIG. 1 and the protocols included in Steps 1, identifying clonotypes of interest requires that the patient undergoes a specific tolerance induction (“STI”) event that either occurs naturally (as in an infarct or flare-up of disease, without limitation intended) or is induced (as in administration of a vaccine or photodynamic therapy, without limitation intended). The STI event is commonly caused by a protocol subjected upon a patient, or correlates approximately in time to the protocol, wherein in different embodiments the protocol can comprise one of more of: (a) injecting weakened Mycobacterium bovis or Mycobacterium tuberculosis as occurs in various vaccines (following protocols known generally as Bacillus Calmette-Guerin) as an adjuvant in treatments for, for example, type 1 diabetes, interstitial cystitis, multiple sclerosis (see, e.g., Ristori et al., NEUROLOGY 53(7):1588-9 (1999)), Parkinson's disease (see, e.g., Yong et al., PLOS ONE 6(1):e16610 (2011)); (b) photodynamic therapy, such as applying psoralen ultraviolet A irradiation (PUVA therapy, for psoriasis) or applying coal tar to psoriatic plaques followed by exposure to ultraviolet B radiation (known commonly as Goeckerman therapy); (c) injecting an amyloid beta vaccine (to treat Alzheimer's disease); (d) injecting an influenza vaccine; (e) dental cleaning or other dental treatment that includes gum trauma; (f) injecting etanercept into the perispinal space (see, e.g., Tobinick, EXPERT REV. NEUROTHER. 10(6):985-1002 (2010); also see Maillot, J. NEURORADIOL. 18(1):18-31 (1991) re definition of “perispinal space”); (g) injecting cortical steroids, etanercept, mesenchymal stem cells, hyaluronic acid, or botulinum toxin into joint space for treating arthritic conditions; (h) manipulations of coronary arteries for angioplasty, stenting, and the like as applied by catheterization methods and devices; and (i) surgery (in general); any of which result in an inflammatory response. Preferably, the protocol employed is (a) injecting weakened Mycobacterium bovis or Mycobacterium tuberculosis, or (b) applying ultraviolet A irradiation.

In other contexts, the STI event is a natural event, which event can be a flare-up of autoimmune disease, such as all-too-commonly occurs with systemic lupus erythematosus, rheumatoid arthritis, and others. Other naturally-occurring STI events include, for example, a myocardial infarct, a brain infarct, a spinal cord injury, or any traumatic event resulting in an inflammatory response by the patient's immunological system. Preferably, the STI event of this category is a flare-up of an autoimmune disease, a myocardial infarct, or a brain infarct.

Both before and after the STI event in most embodiments of the present invention, a lymphocyte-containing sample is collected from the patient. The lymphocyte-containing sample can be any tissue or bodily fluid that contains lymphocytes; preferably, the sample is sourced from a site of autoimmune activity, such as the target organ of an autoimmune disease or site of injury; and particularly in instances of systemic involvement in the autoimmune activity, as in systemic lupus erythrometosus (“SLE”) for example, the sample can be drawn peripheral blood. The pre-STI sample can be collected and stored appropriately, as known in the art, without further processing beyond preparation for storage; alternatively, the pre-STI sample can be collected and processed through preparation of DNA or RNA or cDNA, which can then be stored for several years in precipitated form over alcohol at −20° C., as known in the art. (One protocol for preparation of total RNA through storage over ethanol is presented below; and others are readily available in the relevant literature as known by those of ordinary skill in the art.) Such stored material of the pre-STI sample is a useful precaution for patients afflicted with an autoimmune disease that is characterized by flaring of symptoms or who are at risk for myocardial or brain infarct based on family history and/or general health conditions of weight/height ratios or history of smoking, for example.

The preparation of DNA or RNA (for making cDNA) from a tissue or blood sample can be accomplished using any of many known protocols known to the art. For the purpose of such preparation in the context of the present invention, one would contact the commercial laboratory that is selected to conduct the high throughput sequencing of the DNA or cDNA and use the protocol or protocols of its suggestion for this purpose. For example, one such commercial laboratory, Evrogen JSC (Moscow, Russia) provides a service for profiling TCRs present in a collection lymphocytes (see http://www.evrogen.com/services/service-TCR-profiling/service-TCR-profiling.shtml). Based on Evrogen's recommendations, one harvests total RNA from the lymphocyte-containing sample following the well-known protocol of Chomczynski and Sacchi (ANAL. BIOCHEM. 162:156-159 (1987)), except that all steps are performed at neutral pH instead of acidic and the prepared total RNA is precipitated with lithium chloride (LiCl). Briefly, the Evrogen-recommended total RNA isolation protocol involves the following steps:
(1) dissolving 10 to 100 μl of the pre- or post-STI sample in a 5× or greater volume of buffer in a microcentrifuge tube, where the buffer is composed of 4M guanidine thiocyanate, 30 mM disodium citrate, 30 mM (3-mercaptoethanol, pH 7.0-7.5, using sterile water;
(2) spinning the dissolved sample at maximum speed on a microcentrifuge for 5 minutes at room temperature, and transferring the supernatant to a new microcentrifuge tube;
(3) place tube on ice, add an equal volume of buffer-saturated phenol and mix; then add ⅕ volume of chloroform-isoamyl alcohol (24:1) and vortex 4-5 times with one minute on ice between vortexing; spin on microcentrifuge for 30 minutes at 4° C.; remove the upper, aqueous phase and repeat this step; after the repeat, remove and save the upper, aqueous phase;
(4) add 1 μl of glycogen or a “co-precipitant” product sold by Amersham, and then add 96% ethanol and mix; spin immediately at room temperature on the microcentrifuge for 10 minutes; wash pellet (even if not visible) once with 0.5 ml 80% ethanol; dry the pellet briefly until no liquid is seen in the tube;
(5) dissolve pellet in 100 μl fresh sterile water (e.g., water freshly dispensed from a Milli-Q water filtration station (sold by Millipore Corporation (Billerica, Mass.)) (if pellet does not dissolve completely, spin on microcentrifuge for 3 minutes at room temperature and transfer the supernatant to a new tube); add equal volume of 12 M LiCl and chill at −20° C. for 30 minutes; spin on microcentrifuge for 15 minutes at room temperature; wash the pellet once with 0.5 ml 80% ethanol, and dry as above (precipitated RNA is usually no visible);
(6) add 0.1 vol. of 3 M sodium acetate and 2.5 vol. 96% ethanol to the RNA, mix thoroughly.
   The RNA sample may be stored at −20° C. for several years.

Outside service providers also provide services for generating a cDNA library or, more fundamentally, taking a lymphocyte-containing sample that is flash frozen using liquid nitrogen and then transported to the contracting laboratory under dry ice for further processing using standard protocols to isolate RNA, prepare cDNA, sequence same using primers appropriate to TCR and/or BCR profiling and so generate a profile of receptors found in the provided sample. Once such data is available, one can analyze same to identify newly arising receptors both qualitatively and quantitatively and so identify the prevalent receptors derived from the post-STI sample relative to receptors included in the pre-STI sample. With the prevalent receptors so identified, one can use contract research organizations that have genetic engineering and cell culturing capabilities to generate populations of autologous and/or allogeneic regulatory lymphocytes that express the selected prevalent receptors or chimeric variants thereof that have enhanced capabilities in accordance with the present invention. Or, one of ordinary skill in the relevant art can follow the teachings of this disclosure to generate the materials and practice the inventive method.

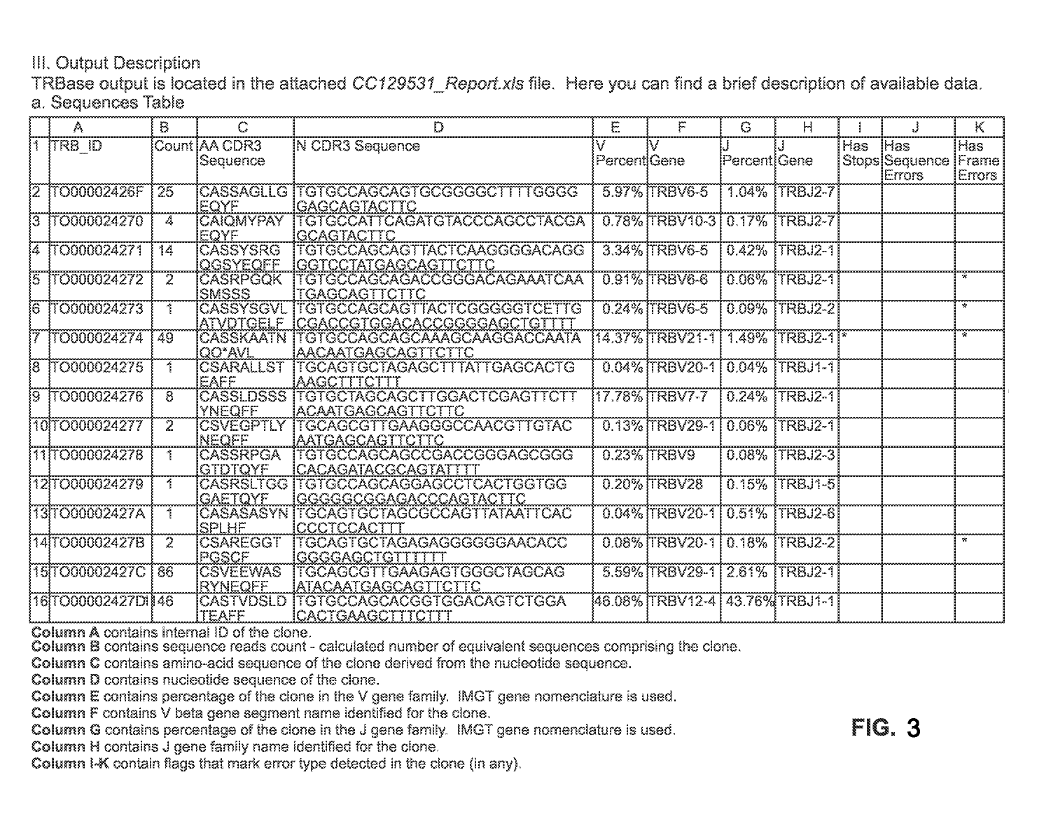

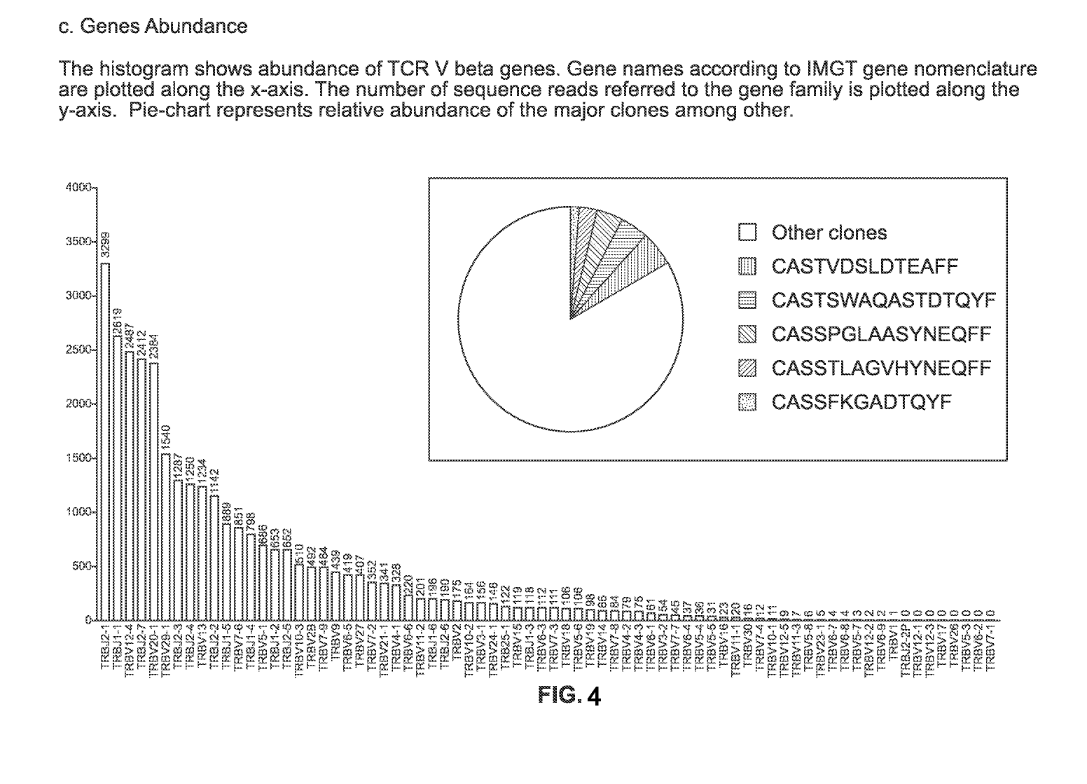

Providers of services for processing a lymphocyte-containing tissue sample and high throughput sequencing of the DNA or RNA present in such samples include the aforementioned Evrogen laboratory located in Moscow, Russia; Adaptive Biotechnologies Corporation of Seattle, Wash. (http://www.adaptivebiotech.com/); Sequenta, Inc. of South San Francisco, Calif. (http://sequentainc.com/); iRepertoire, Inc. of Huntsville, Ala. (http://irepertoire.com/); among others. The data provided by such service providers (examples of which are displayed as FIGS. 3-5 hereof) allows for identification of the prevalent T cell receptors or prevalent B cell receptors (collectively, “prevalent receptors”). FIGS. 3 and 4 show that the TCR beta chain complementarity-determining region 3 (CDR3) sequences and clonotype frequencies are commercially available. (As shown from Evrogen Lab (Moscow, Russia).)

More particularly, FIG. 3 is an exemplary report showing, among other things, a listing of clonotypes (clones), their sequence and read count, the percentage of the clone in the V gene family, the percentage of the clonotypes in the J gene family.

FIG. 4 shows the abundance of T cell receptor V beta genes depicted as a histogram and a pie chart. FIGS. 3 and 4 are examples of computer-based methods that can be used to identify lymphocyte receptor segments that increase in abundance following a specific tolerance induction event.

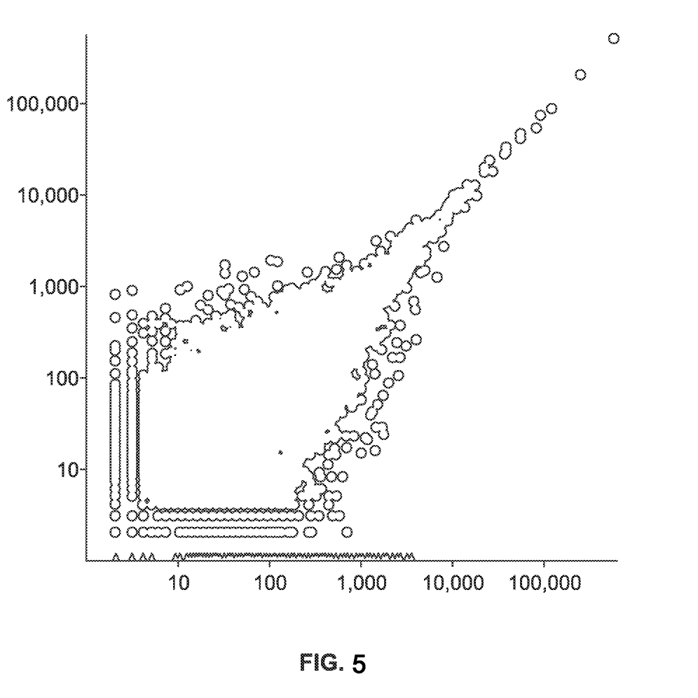

FIG. 5 shows a scatter plot demonstrating the reproducibility of the method. In this case, both the x-axis and y-axis are the same logarithmic scale plotting the same data, so perfectly reproducible data would fall on a 45 degree line ascending from the bottom left to the top right of the graph. FIG. 5 shows that a majority of the data points follow this 45 degree trend of reproducibility.

Referring to FIG. 2, a method for determining T cells or B cells induced or expanded by a patient's immune system upon treatment of an autoimmune disease is exemplified. The figure presents a protocol that can be employed with respect to any autoimmune disease or any injury whose onset or subsequent treatment results in a specific tolerance induction event. Accordingly, although we will present the protocol of the present invention in the context of treatment of a patient afflicted with a specific autoimmune disorder, this description is absolutely not intended to be limiting to an autoimmunity treatment only, or to a disease treatment only for that matter, but is usefully considered as description of one embodiment of the inventive method that can be analogously employed with regard to any disease or injury that induces a specific tolerance induction event or that, upon treatment thereof, induces a specific tolerance induction event that is directed at an immunological attack on self.

In short, the protocol presents a method to identify the response of the patient's immune system and then augment that line of attack to achieve a clinical effect resulting in cure or reduced symptoms.

Further regarding the content of FIG. 1, described above, or FIG. 2, the recital there of a series of steps relates to just one embodiment or a series of related embodiments of the present invention. FIG. 2, for example, is provided here as a tool for discussing one way to practice the invention. However, it is not the only embodiment of the invention. For example, we have determined that the lymphocyte purification step is optional because tools are available that allow the following steps to proceed without difficulty, as is known in the art. Other steps can vary as well, as one can perceive in viewing the structure of our claims below, where fewer than all of the steps set forth in FIG. 1 or 2 are included in our broader claims, which claims as recited describe a completely operative invention. Neither is the order of the steps as set forth in FIG. 1 or 2 fixed. Logic dictates, of course, that collection of a lymphocyte-containing sample, such as a blood or other lymphocyte-containing sample, from prior to a specific tolerance induction event necessarily is collected prior to collection of a lymphocyte-containing sample from after the specific tolerance induction event. But analyzing the included TCRs or BCRs or V/J segments in the earlier sample need not be done until it is needed for comparing to the analogous result from the later sample.

The chart set forth in FIG. 2 describes in vitro (Steps I and II) and in vivo (Steps III) aspects of one embodiment of the inventive treatment. In some embodiments, a population of patients are selected for study that statistically represent the patient population as a whole and/or a subset of the patient population suitable for treatment using the methods described herein. In the first step (i.e., Step I-1), one or more samples of tissue is collected from the patient, if possible; samples of choice are those that contain lymphocytes, such as lymph or blood or a tissue that is subject to autoimmune activity. The lymphocyte-containing tissue sample is taken from a patient afflicted with an autoimmune disease prior to inducing a specific tolerance induction (“STI”) event, such as administering a vaccine or a pharmaceutical directed at reducing the effect of the autoreactive lymphatic cells (also referred to herein as effector cells) that are attacking the autoimmune target tissue. (With respect to a patient who has an accident resulting in, for example, a spinal cord or brain injury, or experiences a flaring of an existing autoimmune disease or a brain or heart infarct, any of which is or results in a proximate (in time) STI event, a pre-STI event tissue sample may not be possible. If a pre-STI event blood sample is available, however, it will serve to heighten the likelihood of success of the treatment, but the lack of such an example should not preclude ability to use the treatment method of the present invention. The present invention can be practiced nonetheless following protocols set forth herein.)

The pre-STI event tissue samples may be analyzed immediately or preserved for later analysis using any suitable method known to the art that preserves the integrity of the contained cells so that a later access of the contained genomic DNA can be subjected to DNA sequencing and analysis.

In a second step (i.e., Step I-2), the patient(s) are treated, meaning that a vaccine or drug is administered that has known impact upon the autoreactive immune cells that are attacking the patient's tissue or tissues. Without being limited to any particular theory, the vaccine or pharmaceutical intervention results in a change in the population of lymphocytes. The lymphocytes include T cells displaying various T cell receptors (TCRs) as well as B cells displaying various B cell receptors (BCRs). In some embodiments, TCRs or BCRs that are specific to the autoimmune-attacked tissue can be utilized in methods of treatment. In some embodiments, samples of the attacked tissue is analyzed immediately or preserved for later analysis. Methods of analysis can include methods described herein or any suitable method of genetic and/or biochemical analysis known to those skilled in the art.

In other embodiments, the tissues are preserved, optionally as formalin-fixed, paraffin embedded (FFPE) tissue.

In a third step of the method depicted in FIG. 2 (Step I-3), another lymphocyte-containing tissue sample is collected from the patient at one or more times following the STI event. Any time period may be suitable and may be adjusted to coincide with the timing of an immunological response in the patient. In some embodiments, the lymphocyte-containing samples are collected at a plurality of times. In other embodiments, the lymphocyte-containing samples are collected on the same day as intervention, and again at 2 days, 3 days, 5 days, 7 days, 10 days, 14 days, 21 days, 30 days, and the like following surgery. The post-STI event blood samples may be analyzed immediately or preserved for later analysis.

In Step 4 of FIG. 2 (i.e., Step I-4), lymphocytes are separated and purified from the collected tissue. One example of a method of lymphocyte separation from a blood draw comprises layering heparinized venous blood onto a density gradient of Ficoll-Isopaque (GE Healthcare Biosciences, Pittsburgh, Pa.) in 15 ml conical tube in the ratio 3:1; centrifuging the tube at about 1800 rpm for about 20 minutes; removing the middle layer; transferring to another tube; centrifuging the tube at about 2000 rpm for about 5 minutes to about 15 minutes; discarding the supernatant; suspending the pellet in RPMI-1640 medium (Life Technologies Corporation, Grand Island, N.Y.); washing the cells twice with the RPMI-1640 medium; suspending the cells in about 1 ml of the RPMI-1640 medium; dividing the cell suspension into two tubes; incubating the suspensions with fluorescent reagents such as monoclonal antibodies specific for CD4 or CD8 cells, which antibodies are tagged with a fluorochrome, such as phycoerythrin (PE) or 5(6)-fluorescein isothiocyanate (FITC) (BD Biosciences Pharmingen, San Diego, Calif.); vortexing the suspension at room temperature; incubating the suspension for 2 hours in the dark at room temperature; and identifying the specific lymphocytes based on staining with specific antibodies for sorting on a cell sorter apparatus such as the BD FACStar Plus (BD Biosciences, San Jose, Calif.). One protocol for separating and purifying the lymphocytes is set forth in Toor and Vohra, MICROBES INFECT. 14(12):1111-7 (2012). This protocol can be applied to lymph-derived or solid tissue samples as well, although the solid tissue sample would have to be teased apart in a saline solution prior to the first step.

Separation and purification can also be accomplished by immunomagnetic selection with microbeads. The immunomagnetic selection can be done with commercially available kits such as Miltenyi CD8+ kits (Miltenyi Biotec Inc., Auburn, Calif.). Other separation methods such as fluorescence-activated cell sorting (FACS) can also be used to separate and purify lymphocytes from collected samples in accordance with methods generally known to those of ordinary skill in the art, including the methods set forth in U.S. Patent Application 2011/0300119, which is hereby incorporated herein by reference, especially with respect to Paragraphs [0064] to [0072] and [0089] to [0090] thereof.

Lymphocyte isolation may be done using positive isolation, negative isolation, or a sequence of steps that includes both positive and negative isolation. For example, CD3+ T cells can be separated using an Invitrogen Dynabeads CD3 kit (Catalog #11151D or 11365D, available from Life Technologies Corporation, Grand Island, N.Y.), or the CD3 MicroBeads or CD56 MultiSort Kit from Miltenyi Biotec Inc. (Auburn, Calif.), or other such kits designed for isolation of other lymphocytes; other suitable separation methods can also be used. For non-magnetic separations, a Pluriselect kit (Catalog #10-00300-21, Pluriselect GmbH, Leipzig, Germany) may be used. Kits from the mentioned manufacturers and other manufacturers may be used for negative selection. In addition, more automated tools such as the Miltenyi Biotec autoMACS Pro Separator (Miltenyi Biotec Inc., Auburn, Calif.) may be used.

In Step I-4 of FIG. 2, DNA is isolated from any one or more of the pre-STI event lymphocyte samples. In some embodiments, DNA is extracted from a mixed tissue or mixed cell-type sample, optionally from whole blood. This embodiment may eliminate the need for certain sample processing steps, whereby the genetic loci of interest can be interrogated from a mixed DNA sample. In another embodiment, the blood and/or tissue samples are first enriched for certain lymphocytes, optionally by whole blood fractionation. Whether from an enriched sample, or from a non-enriched sample, DNA can be isolated according to any suitable method known to those skilled in the art. Ones skilled in the art will be aware of DNA prep kits such as QiA DNA Blood Maxi prep #51192 or QIAGEN DNeasy Blood and Tissue kit #69506. Complementary DNA (cDNA) may also be prepared with kits such as QIAGEN FastLane Cell cDNA Kit #215011 using isolated RNA. The identified kits are available commercially from QIAGEN Inc. (Sunnyvale, Calif.).

Step I-4 of FIG. 2 may include amplification of the extracted DNA (if necessary) and sequenced; alternatively, Step I-4 can include spectratype analysis as further described below. Specifically, the DNA encoding the lymphocyte receptors is amplified in some embodiments, optionally the T cell receptors. T cell receptors consist of alpha (α) and beta (β) chains. In some embodiments, both the alpha and beta chains of a TCR are amplified. To amplify a section of DNA using PCR, one requires two primers. Primers are short strands of DNA that physically stick (or anneal) to a segment of the DNA, and allow other molecules (known as polymerases) to “copy” what is between them. In other embodiments, various loci can be amplified separately. For example, the alpha and beta chains of a TCR can be amplified separately to yield two PCR products. Skilled persons will be familiar with methods in polymerase chain reaction (“PCR”) that are suitable for DNA amplification including design of short, single-stranded pieces of DNA that serve as PCR primers, adjustment of annealing, melting and extension times and temperatures and the like such that high quality PCR products are produced. Some DNA amplification procedures may be conducted in multi-well plates.

Step I-4 of FIG. 2 includes DNA sequencing of the DNA. Methods of DNA sequencing are well known in the art and have improved rapidly in recent years in features such as read length, improved throughput, reduced cost and the like. One suitable method of DNA sequencing is pyrosequencing. Pyrosequencing is a method of DNA sequencing (determining the order of nucleotides in DNA) based on the “sequencing by synthesis” principle. It differs from Sanger sequencing, in that it relies on the detection of pyrophosphate release on nucleotide incorporation, rather than chain termination with dideoxynucleotides as used in the Sanger method. DNA sequencing results in sequence data of adenine (A), cytosine (C), thymine (T) and guanine (G) that is analyzed by computer methods.

Step I-5 includes methods for identifying lymphocytes and/or lymphocyte receptor sequences that have expanded following STI event. For most locations along a cell's DNA, the sequence is identical from cell to cell. However, the T cell receptor and B cell receptor locations are different. DNA regions coding for T cell receptor and B cell receptor, the body uses a process known as somatic or “VDJ” recombination to recombine the DNA at that location (in one individual cell). Therefore, if one takes a population of T cells or B cells (from a blood draw, for example, or from other tissue that includes lymphocytes), and carries out a targeted resequencing of the DNA at the T cell or B cell receptor locus, one will get many different “reads”—or sequences of DNA (each read comes from a different cell).

When performing targeted resequencing, a mixture of different primers is used. This provides for “multiplexed” PCR that results in amplification of a series of different DNA sequences. The primers correspond to the DNA at either end of the DNA segment targeted for amplification. In this way, the primers act as “bookends.” The bookends vary from cell to cell, but the possible bookends come from a small, fixed set. In the case of T cell and B cell receptors, there are known bookends called V segments and C (constant) segments; by performing resequencing using a mixture of primers, all the possibilities are covered. Every T cell receptor or B cell receptor will include one V segment and one C segment. The DNA between the bookends is amplified and the amplified DNA may then be sequenced using well known techniques such as pyrosequencing.

Methods for conducting repertoire audits of a subject have been published including, for example, U.S. Patent Application 2005/0064421, which is incorporated herein by reference.

These sequences represent a “repertoire” which can be analyzed. In particular, one may decipher which sequences are more or less common. In addition, one may take two tissue or blood samples (“before” and “after” the STI event) and decipher which sequences were absent before the STI event but appeared thereafter. By “expansion” it is meant that the number of members of a clonotype is greater relative to other clonotypes found in the same blood sample following the medical intervention or injurious event that induces an STI event; as compared to the clonotypes identified in a blood sample collected from the same patient before the STI event. Expansion can be quantified by comparing the amount of amplified DNA for a given alpha or beta chain from samples before and after the STI event.

The methods of Step I-5 are often computer-based. In some embodiments, clonotypes are considered highly expanded if their frequency (in the measured repertoire) is about 0.5% units or greater. In some embodiments, a clonotype which is absent or not highly expanded prior to the STI event, but which is highly expanded after it, is inferred to be an autoimmune-associated clonotype. In some embodiments, a clonotype will be inferred to be a specific autoimmune clonotype if it is highly expanded both before and after the STI event, but has a frequency that increases from before to after the STI event, where the increase is statistically significant using an appropriate multiple hypothesis testing statistical method to stringently limit the false discovery rate.

In other embodiments, sequencing is replaced with spetratyping (also known as immunoscoping). Spectratype analysis takes advantage of PCR technology to amplify template cDNA corresponding to rearranged transcripts with different complementarity-determining region 3 (CDR3) lengths from specific TCR variable region genes in a competitive manner. Persons of ordinary skill in the art are familiar with protocols for spectratype analysis, such as those available at http://www.currentprotocols.com/WileyCDA/CPUnit/refId-im1028.html.

When treating a patient by the instant inventive method using spectratype analysis, the spectratype analysis of a patient's lymphocytes is carried out before and after a specific tolerance induction event, such as a pharmaceutical intervention or injury event, to identify the particular lymphocyte receptor V or J segments that expanded subsequent to the specific tolerance induction event. A subset of cells expressing the identified V and/or J segments can then be isolated from the patient's own blood, expanded, stimulated, or modified ex vivo, and then reinfused as a therapeutic treatment.

It is understood that FIG. 2 is a depiction of generalized procedures provided to illustrate several embodiments of the present invention and is not to be considered limiting in any way. For example, Step I-4 of FIG. 2 does not limit the method of DNA extraction, amplification or sequencing simultaneously employed in the context of the present invention. Furthermore, there may be other steps not necessarily depicted in the figures, such as sample processing and the like; and the listing of steps in a given order is not in and of itself a recital that the identified steps must be accomplished in that order, irrespective of how they are listed or described in the detailed description, figures, examples, or, for that matter, claims. For example, one of ordinary skill necessarily understands that Steps I-4 and I-5 comprising the steps of: purification; DNA isolation; amplification; sequencing; and identification of lymphocyte clonotypes may be carried out immediately after each blood draw or blood samples drawn or tissues collected before a specific tolerance induction event can be stored and processed per steps I-4 and I-5 at the same time that a blood sample is drawn or tissue sample collected subsequent to the STI event.

In some embodiments, Step I-5 of FIG. 2 results in separate data comprising alpha chains that are induced upon vaccination and beta chains that are induced upon pharmaceutical intervention.

Step I-6 of the procedure depicted in FIG. 2 involves “paired chain analysis.” In this step, various methods can be utilized to pair induced alpha and beta chains such that the pairing results in a TCR or BCR that binds to an epitope of the tissue subject to the autoimmune attack. In some embodiments, post-sequencing pairing may be unnecessary or relatively simple, for example in embodiments in which the alpha and beta chain pairing information is not lost in the procedure, such as if one were to sequence from single cells.

In some embodiments, the chain pairing is assisted in silico by computer methods for annotating VDJ gene segments. For example, specialized, publicly-available immunology-directed gene alignment software is available from International IMmunoGeneTics information system (IMGT; Lefranc et al., NUCL. ACIDS RES. 37:D1006-D1012 (2009)), including V-Quest (Brochet et al., NUCL. ACIDS RES. 36:W503-508 (2008)) and JunctionAnalysis (Yousfi Monod et al., BIOINFORMATICS 20:i379-i385 (2004)); JOINSOLVER (Souto-Carneiro et al., J. IMMUNOL. 172(11):6790-6802 (2004)); VDJSolver (Ohm-Laursen et al., IMMUNOLOGY 119(2):265-77 (2006)); Somatic Diversification Analysis (SoDA; Volpe et al., BIOINFORMATICS 22(4):438-44 (2006)); iHMMune-align (Gaeta et al., BIOINFORMATICS 23:1580-1587 (2007)), and other similar tools.

In some embodiments, the chain pairing is done using VDJ antibodies. For example, one may obtain antibodies for the identified segments and use the antibodies to purify a subset of cells which express that gene segment in their (surface) receptors (e.g., using FACS, or immunomagnetic selection with microbeads). One may then sequence from this subset of cells which have been purified for the desired gene segments. If necessary, this secondary sequencing may be done more deeply (i.e., at a higher resolution) than the first round of sequencing. In this second sequence data set, there will be far fewer induced clonotypes, greatly easing the task of chain pairing. Depending on the gene segments, there may be only one induced alpha chain and one induced beta chain for example.

In some embodiments, the chain pairing is done by trial and error.

In some embodiments, the chain pairing is done using multiwall sequencing. For example, one may isolate gene segment purified cells or unpurified cells into a microwell plate, where each microwell has a very low number of cells. One can amplify and sequence the cells in each well individually, which provides another means to pair the chains of interest by sequencing on a single cell basis, facilitating the pairing of induced alpha and beta chains.

One skilled in the art necessarily understands that purification of lymphocytes may be performed after each collection of lymphocyte-containing tissue or may be performed after the last collection of lymphocyte-containing tissue has been completed. Moreover, identification of lymphocyte clonotypes may also follow after each collection of lymphocyte-containing tissue or after the completion of the intended collections of lymphocyte-containing tissue with respect to the analysis surrounding the specific tolerance induction event.

Step II-1 of FIG. 2 includes genetic engineering of autologous or allogeneic T cells or B cells to display the TCR, BCR, or chimeric antibody receptor corresponding to the induced clonotype(s). Methods of genetic engineering are generally known in the art and can be found in well-known texts of protocols including Sambrook et al. (2001), MOLECULAR CLONING. A LABORATORY MANUAL, Cold Spring Harbor Press, Plainview, N.Y.), to provide one non-limiting example; also available is a rich patent literature that details protocols usefully employed in the context of this invention, including, for example: U.S. Patent Applications 2010/0047220, 2010/0135974, and 2011/0020308, and U.S. Pat. No. 8,361,794, each of which are incorporated herein by reference.

The alpha and beta chains of the T cells of this invention may be expressed independently in different hosts or in the same host. Preferably the alpha and beta chains are introduced into the same host to allow for formation of a functional T cell receptor in the host cell. In some embodiments, the host cell is capable of inducing an immune response in a patient. The means by which the vector carrying the gene may be introduced into the cell include, but are not limited to, microinjection, electroporation, transduction, retroviral transduction or transfection using DEAE-dextran, lipofection, calcium phosphate, particle bombardment mediated gene transfer or direct injection of nucleic acid sequences encoding the T cell receptors of this invention or other procedures known to one skilled in the art and set forth in well-known and utilized sourcebooks such as Sambrook et al., Id.

Genetic engineering of human T cells may also be accomplished using lentiviral vector gene transfer, as is well-described in the art. Protocols for this method are known to one skilled in the art. See, e.g., Verhoeyen et al., METHODS MOL. BIOL. 506:97-114 (2009).

In some embodiments, a T cell is engineered to display a functional TCR. In other embodiments, a chimeric cell may be engineered in which a T cell displays an alternative type of receptor such as a B cell receptor. See, e.g., U.S. Application 2010/0135974.

In Step II-2 of FIG. 2 includes in vitro assays. In some embodiments, the autologous engineered cells from Step II-1 of FIG. 2 are incubated with autoimmune target tissue or lysate. In various embodiments, various effects are measured during the incubation such as cytokine concentration, cell proliferation, and the like. In some embodiments, the effects of various adjuvants are quantified.

Turning now to Steps III of FIG. 2, depicted herein is an exemplary sequence of steps which comprise a therapeutic strategy. T cell receptors may be applied in a treatment for autoimmune disease using T cell autologous or allogenic adoptive transfer cell therapy. In this method, T cells from the patient (autologous) or a donor (allogeneic) are retargeted by a process of engineering, in which the endogenous T cell receptor is suppressed, and an alternative T cell receptor sequence (as determined previously) is introduced into the cell. These retargeted T cells are reinfused into the patient and monitored for efficacy in destroying the targeted cells. In some embodiments, the cells further include one or more adjuvants suitable to illicit or amplify an immune response.

The cells may be administered for therapeutic purposes in any physiologically acceptable medium, normally intravascularly, although they may also be introduced into bone or other convenient site. Usually, at least 1×10⁵ cells are administered, preferably 1×10⁶ or more. The cells may be introduced by injection, catheter, or the like. The cells may be frozen at liquid nitrogen temperatures and stored for long periods of time, being capable of use on thawing. If frozen, the cells will usually be stored in a 10% dimethyl sulfoxide (“DMSO”), 50% fetal calf serum (“FCS”), 40% RPMI 1640 medium (Sigma-Aldrich, St. Louis, Mo.). Once thawed, the cells may be expanded by use of growth factors and/or stromal cells associated with progenitor cell proliferation and differentiation, as known in the art.

In Steps III, the effects of the treatment are evaluated by reference to clinical and surrogate endpoints. The clinical endpoints include assessment of inflammation and pain associated with the autoimmune target tissue. The surrogate endpoints may be monitored, in the instance of Type 1 diabetes, for example, by testing serum levels of c protein, glycated hemoglobin (HgB A1C), insulin, or glucose. Other surrogate endpoints usefully employed in the context of the present invention include, without limitation intended, monitoring serum levels of C—reactive protein (CRP) to assess inflammation. Considering now the present invention as a whole, the inventive method of treatment of a patient who suffers from an autoimmune disease, wherein the patient undergoes or experiences a specific tolerance induction (“STI”) event, can include variations of the following steps: collecting a first lymphocyte-containing sample from the patient prior to the STI event (“pre-STI event sample”); detecting an STI event or performing a procedure that correlates in time to an STI event; collecting a second lymphocyte-containing sample from the patient after the STI event (“post-STI event sample”); preparing lymphocytes from the pre-STI event sample or the post-STI event sample; preparing and sequencing DNA or cDNA derived from the prepared lymphocytes; identifying sequences of prevalent T cell receptors (“TCRs”) or prevalent B cell receptors (“BCRs”) (collectively, “prevalent receptor sequences”) derived from the post-STI event sample; selecting a regulatory lymphocyte that carries at least one of the prevalent receptor sequences or a sequence that is about 85% identical or greater with respect to one of the prevalent receptor sequences, which selected regulatory lymphocyte (i) expresses one or more prevalent receptor sequences or (ii) is generated from an autologous or allogeneic naïve lymphocyte, which naïve lymphocyte is engineered and induced to become a regulatory lymphocyte that expresses at least a receptor sequence that is about 85% identical or greater with respect to one of the prevalent receptor sequences; culturing the selected regulatory lymphocyte, thereby generating daughter cells of said regulatory lymphocyte; and administering said daughter cells to said patient.

Collecting the pre-STI event sample is considered an optional step in that one can surmise the more prevalent TCRs or BCRs in the post-STI event sample in the absence of having the pre-STI event sample. There are drawbacks associated with not having the pre-STI event sample and its analysis because it can happen that one or more of the more prevalent post-STI event clonotypes could be of the same or highly similar concentrations as found in the patient's pre-STI event sample, and so a clonotype that is among the more prevalent could be indicated for the present method and yet be inoperative when the pre-STI sample analysis is not available. Nonetheless, we view the pre-STI event sample as optional because among the more prevalent clonotypes identified from analysis of the post-STI event sample alone will be at least one clonotype that can be usefully employed to generate an operative therapy under the present invention. For a patient that suffered a spinal cord injury, it may be the case that no pre-injury blood sample may have been collected; but given the benefits of the present invention in reversing or retarding such trauma, it would be a rational plan to grow up the more concentrated T_{reg} or B_{reg} clonotypes and testing them by infusing one at a time, or a subset of them, to attempt amelioration or even reversal of what is commonly today a significant and intractable injury that has virtually no effective treatments available. Of course, it is preferable that the pre-STI sample be available as it reduces the variables in identifying the clonotypes usefully employed for the method of the present invention.

The step of detecting an STI event or performing a procedure that correlates in time to an STI event has been discussed above as to various procedures that are deemed useful for causing the STI event, or correlating in time to such an event (because causation here is not considered proven, nor need it be proven). And whether the act that correlates in time with the STI event is itself a direct or indirect cause is also unknown, and also not needed. The key here is to have defined a point in time before and after which lymphocyte-containing samples are desirably obtained. However, as stated above, only the post-STI sample is absolutely necessary for practicing the inventive method.

If, however, a lymphocyte-containing sample was collected from the patient prior to the STI event (i.e., the first lymphocyte-containing sample), then the step of identifying sequences of prevalent TCRs or BCRs (collectively, prevalent receptors) derived from the lymphocytes of the post-STI event sample (i.e., the second lymphocyte-containing sample) can, in some embodiments, further comprise identifying prevalent TCRs or BCRs from the post-STI event sample relative to TCRs or BCRs, respectively, from the pre-STI event sample. Sometimes the prevalent receptor is unique to the post-STI event sample; and, other times, the prevalent receptor is present in both the pre- and post-STI event samples, but is significantly increased in concentration in the post-versus the pre-STI event sample.

The inventive method can include the step of selecting clonotypes from the post-STI event sample that are not identified among clonotypes from the pre-STI event sample or that have an expansion frequency of about 0.5% or greater relative to the clonotypes from the pre-STI event sample. Part of this embodiment includes the further step of selecting clonotypes from the post-STI event sample that are not identified among clonotypes from the pre-STI event sample or that have an expansion frequency of about 0.5% or greater relative to the clonotypes from the pre-STI event sample.

Another embodiment provides a method further comprising selecting clonotypes as highly expanded or newly abundant if their frequency (in the measured repertoire) has increased by a level of about 0.1% or greater, about 0.2% or greater, about 0.3% or greater, about 0.4% or greater, about 0.5% or greater, about 0.6% or greater, about 0.7% or greater, or about 0.8% or greater.

Another embodiment provides a method further comprising selecting a clonotype that is absent or not highly expanded prior to a medical procedure that is or approximately correlates in time to the STI event, but which is highly expanded or abundant relative to other clonotypes in the patient's lymphocyte-containing sample collected after the medical procedure, which selected clonotype is referred to as a treatment-associated clonotype.

Another embodiment provides a method further comprising selecting a clonotype that is absent or having a concentration that is not highly expanded, meaning its concentration is about average or less relative to other clonotypes of the lymphocyte population prior to a medical procedure, but which is highly expanded or abundant relative to other clonotypes in the patient's lymphocyte-containing sample collected after the procedure, which selected clonotype is referred to as a treatment-associated clonotype.

Another embodiment provides a method further comprising selecting a clonotype that is absent or having a concentration that is not highly expanded, meaning its concentration is about average or less relative to other clonotypes of the lymphocyte population prior to the specific tolerance induction event, but which is highly expanded or abundant relative to other clonotypes in the patient's blood sample drawn after the specific tolerance induction event such that its relative concentration is then at least about 3%, or at least about 4%, or at least about 5%, or at least about 6%, or at least about 7%, or at least about 8% with respect to the other clonotypes of the post-STI event population of lymphocytes; which selected clonotype is referred to as a treatment-associated clonotype.

Another embodiment provides a method further comprising selecting a clonotype as an autoimmune-specific clonotype if it is highly expanded both before and after an STI event, but has a frequency that increases from before to after the STI event that correlates to reduced inflammation in the autoimmune target tissue, wherein the increase in frequency is statistically significant using an appropriate multiple hypothesis testing statistical method to limit the false discovery rate stringently.

Another embodiment provides a method further comprising selecting a clonotype as an autoimmune-specific clonotype if it is highly expanded both before and after an STI event, but has a frequency that increases from before to after the STI event that correlates to reduced inflammation in the autoimmune target tissue, wherein the increase in frequency is statistically significant using an appropriate multiple hypothesis testing statistical method to limit the false discovery rate stringently; wherein, in some embodiments, the pre-STI event relative frequency of the clonotype is at least about 3% and the post-STI event frequency, with respect to its pre-STI event frequency, is about 3% points higher, or about 4% points higher, or about 5% points higher, or about 6% points higher, or about 7% points higher, or about 8% points higher.

The selection of clonotypes comprises spectratype analysis or DNA sequencing or both. Such selection commonly includes further steps of (a) identifying a set of one or more V or J segments among the sequenced DNA whose prevalence increased when comparing the pre-STI event sample to the post-STI event sample, i.e., the prevalent receptor sequences; and (b) isolating or selecting a regulatory lymphocyte bearing the identified V or J segment(s), as one example.

The method set forth herein below is amenable wherein the autologous regulatory lymphocyte is a regulatory T cell (“T_{reg} cell”) or a regulatory B cell (“B_{reg} cell”); in one embodiment, the autologous regulatory lymphocyte employed is a T_{reg} cell; in another embodiment, the autologous regulatory lymphocyte employed is a B_{reg} cell; in yet a further embodiment, the method employs both a T_{reg} cell and a B_{reg} cell.

In another embodiment, the regulatory lymphocyte is selected by identification of certain genetic markers on the lymphocytes' surface, which markers include one or more of CD4, CD25, CD19, CD5, FoxP3. For example, CD4⁺, CD25⁺ T cells are identified as regulatory T cells. See Sakaguchi et al., IMMUNOL. REV. 212:8-27 (2006). Similarly, CD19⁺, CD5⁺, FoxP3⁺ B cells have been identified as regulatory B cells. Noh et al., CELLULAR IMMUNOLOGY 274:109-114 (2012).

In yet another embodiment, the selected regulatory lymphocyte is a T_{reg} cell. The T_{reg} cell, in certain embodiments, includes the genetic markers CD4⁺, CD25⁺, or FoxP3⁺ and one or more further genetic markers selected from the group consisting of CD25^{Hi}, CD197+, CD194+, CD103⁺, Folr4⁺/FR4⁺, Nrp1⁺, HLA-DR⁺, CD279⁺, CD134⁺, CD137⁺, LAP⁺, GITR⁺, CD152⁺, CD45RA⁺, and CD127^{low}.

One embodiment category employs regulatory lymphocytes that are B_{reg} cells. In certain such embodiments, the regulatory lymphocytes include CD5⁺ and CD1d⁺ and one or more genetic markers selected from the group consisting of TIM-1⁺, IL-10⁺, CD21^{hi}, CD23⁺, CD24⁺, CD27⁺, and CD38⁺.

By analysis of the genetic markers, and sectoring the cells into separate sets, one of ordinary skill in the immunological arts can readily identify sets of autologous cells that are predominantly B_{reg} cells or T_{reg} cells. Antibodies directed at the various cell surface receptors of the various lymphocytes can be purchased from a variety of biological supply houses; numerous technical articles have been published that report cell surface antigens that are characteristic of T_{reg} cells and B_{reg} cells, respectively; and numerous technical articles also set forth methods for sorting cells using such antibodies of identified and characteristic cell surface antigens.

One more selection step improves the so obtained set of predominantly B_{reg} cells or T_{reg} cells, which is to select for cells having the same BCRs or TCRs, respectively. As further set forth herein, one can isolate B_{reg} cells or T_{reg} cells that are uniform with respect to their class receptors by VDJ analysis. Because VDJ analysis is protein based, one can obtain antibodies specific to the particular V or J portion included in the identified B_{reg} cells or T_{reg} cells and, using the same cell sorting techniques referred to above, isolate a subset of the regulatory lymphocytes that are specific for the same autoreactive T cell, for example.

Once having isolated a set of lymphocytes that are predominantly T_{reg} cells or B_{reg} cells, or, going the further step, are predominantly specific for a particular autoreactive T cell (with respect to the so-isolated T_{reg} cells) or T_{reg} (with respect to the so-isolated B_{reg} cells), the lymphocyte set is cultured in vitro to generate identical daughter cells and thereby expand the number of cells in the culture. Once so cultured, the expanded culture of regulatory lymphatic cells is quantified for formulating a therapeutic composition (the aforementioned “cocktail”) that includes the autologous lymphatic cells and then introduced back into the patient. The therapeutic composition of expanded lymphocytes can be administered per se or in combination with other active ingredients, such as one or more cytokines. The administration of the therapeutic composition is commonly accomplished by known infusion techniques.

The therapeutic composition can include a biological drug or small molecule drug that suppresses the patient's immune response. Some embodiments that employ this additional step include one or more biological drugs or small molecule drugs that are selected from the group consisting of: a corticosteroid, such as prednisolone or hydrocortisone, without limitation intended; a calcineurin inhibitor, such as cyclosporine or tacrolimus, without limitation intended; an anti-proliferative, such as azathioprine, cyclophosphamide, methotrexate or mycophenolate, without limitation intended; an mTOR inhibitor, such as sirolimus or everolimus, without limitation intended; an anti-inflammatory, such as a tumor necrosis factor alpha (“TNFα”) blocker, without limitation intended; an anti-T cell antibody, such as anti-thymocyte globulin, anti-lymphocyte globulin, Rituximab (a monoclonal anti-CD₂₀ antibody), Basiliximab and Daclizumab (monoclonal anti-IL-2Rα antibodies), without limitation intended; and a tumor necrosis factor antagonist, such as etanercept, without limitation intended. Preferably, the biological drug or small molecule drug is selected from the group consisting of a TNFα blocker, etanercept, an anti-T cell antibody, azathioprine, cyclophosphamide, methotrexate, mycophenolate, sirolimus, tacrolimus, and a cytokine.

In another embodiment of the present invention, the selected regulatory lymphocytes are enhanced by adding additional genetic information for expressing a heightened concentration of T cell receptors (“TCRs”) or B cell receptors (“BCRs”) per T_{reg} cell or B_{reg} cell, respectively, or a heightened concentration of T_{reg} cells or B_{reg} cells that express approximately the same level of TCRs and BCRs as the patient's native such cells, as appropriate.

In yet another embodiment of the present invention, naïve lymphocytes are isolated from the patient, genetically engineered to express certain TCRs or BCRs that are identified to be of a clonotype of heightened concentration subsequent to a specific tolerance induction (“STI”) event relative to the clonotypes that exist in blood samples from the same patient from prior to the STI event, as appropriate, induced to become a regulatory lymphocyte using protocols available and known to skilled artisans, expanded in numbers in culture to create a set of daughter cells that are regulatory lymphocytes that express the TCRs or BCRs identified from the clonotype analysis of the patient's post-STI blood, and then the daughter cells are infused into the patient using methods well known in the art. Protocols are readily available from the literature, including, e.g., U.S. Patent Application 2012/0058096, which is incorporated herein by reference.

In one embodiment, the relative concentrations of the various clonotypes of regulatory lymphocytes is determined from the lymphocyte-containing sample drawn after an STI event. One approach focuses on the use of flow cytometry to sort one of the regulatory lymphocyte classes from others, namely the regulatory T cells (“T_{reg} cells”). See, e.g., Brusko et al., IMMUNOL. REV. 223:371-390 (2008). A combination of genetic markers are known to be expressed on many T_{reg} cells, such as, for example, CD4⁺, CD25⁺, and CD127^{low/-} (which is the interleuken-7 (“IL-7”) receptor α-chain). The concentration of the CD127 marker is known to be inversely correlated to the concentration of FOXP3 expression in human cells, which genetic marker is strongly correlated to T_{reg} cells. Liu et al., J. EXP. MED. 203:1701-1711 (2006). Brusko et al. have shown that this approach yields a cell fraction that is highly enriched for T_{reg} cells, over 95% of which express FOXP3. Brusko et al. at 381.

T cells that express the CD25 marker can be non-regulatory cells. To exclude such non-regulatory CD25⁺ cells, one can further select and remove from the cell fraction those cells that express CD45RA, as demonstrated in Hoffmann et al., BLOOD 108:4260-4267 (2006). The CD45RA⁺ cells are naïve, i.e., not yet regulatory cells, but which will develop into T_{reg} cells in culture, and will also be a highly enriched fraction of cells with respect to FOXP3 expression.

Another protocol usefully employed to isolate regulatory T cells involves selecting for T cells that express a particular TCR and that also express certain markers indicative of regulatory T cells (“T_{reg} cells”), e.g., CD4⁺ and FoxP3^{Hi} using in vitro cell culture and fluorescence-activated cell sorting (“FACS”), as known in the art. See Ellis et al., J. VIS. EXP. 62:e3738 (2012). Such T_{reg} cells selected in this fashion, which is further set forth below in an example, can be then cultured in vitro to raise, for example, a billion or so daughter cells that can then be injected into the very patient from whom the progenitor cells were taken in the first place.

The most prevalent clonotypes included in the fraction of cells that are enriched for T_{reg} cells are identified by their included TCRs, which are identified by sequencing the DNA isolated from this fraction of cells. This clonotype data is used for generating induced regulatory lymphocytes having one or more of the so-identified receptor sequences. Alternatively, or in addition depending on the embodiment of the present invention employed, the identified receptor sequences are used in a standard cell sorting protocol as described above. The isolated set of regulatory lymphocytes is then grown in vitro to expand the number of such cells by growing the aforementioned daughter cells, after which the expanded cells are injected into the patient.

In another embodiment of the present invention, the most prevalent clonotypes identified in the patient's lymphocyte-containing sample collected after the STI event are compared to the clonotypes from a lymphocyte-containing sample collected from the patient that occurred prior to the STI event. Noting that the most prevalent clonotypes from after the STI event are either absent or of a low concentration among the clonotypes present prior to the STI event is a necessary condition for accepting a clonotype for expansion and injection into the patient. In another embodiment that is further discussed elsewhere herein, clonotypes are selected that have increased in concentration by at least 0.1% of the total clonotypes present in post-STI blood relative to the relative concentration of the same clonotype found in the pre-STI blood. Such an observation of the body's policing cells is consistent with the concept that the newly increased concentration of clonotypes were assembled to down-regulate the inflammatory autoreactive T cells (“Tₐᵤₜₒ cells”) that have been attacking the patient's own tissue, irrespective whether the clonotypes of greater concentration are unique to the post-STI blood.

At one level, regarding one embodiment of the present invention, the adoptive cell therapy that is the focus of the present invention is boosted generally by administering suitable cytokines known to down-regulate an immune response in suitable combinations at a time point that is proximate to the occurrence of an action that results in an STI event. At a second level, regarding another embodiment of the present invention, that effort is boosted specifically by administering to a patient a heightened concentration of autologous lymphocytes that share the TCR or BCR or an optimized engineered form the TCR or BCR of the more abundant lymphocyte clonotypes that arose in the patient's blood subsequent to the STI event.

In one embodiment, the present invention comprises a procedure, and sequencing of the repertoire of receptors associated with the lymphocytes from a patient's blood after the procedure, or, in another embodiment, both before and after the procedure, in order to detect and sequence the alpha and beta loci of TCRs and BCRs that relate to highly expanded T cell and B cell clonotypes, respectively, found in the blood.

An important and very helpful variation on selection of source of lymphocytes to be studied in the context of the inventive method that is in accordance with another embodiment of the invention is to assess the TCR and BCR repertoire changes before and after a specific tolerance induction event in the bone marrow, which is readily accessed using standard bone marrow aspirations. Obviously, there will be overlap of information derived from the marrow as compared to the cell population circulating in the blood; importantly, the results from a marrow study will also provide information relating to the prognosis of the disease in the patient.

In some embodiments, the differential information relating to the receptor abundancies among lymphocytes is used to create autologous genetically engineered T cells and/or B cells with lymphocyte receptors found in native regulatory lymphocytes that target the autoreactive T cells focused on the object tissue of the autoimmune disease of interest. Additionally, engineered receptors can also be introduced to autologous cells, induced to become regulatory lymphocytes, and infused into the patient.

Finally, for those embodiments that rely on creating engineered autologous or allogeneic regulatory lymphocytes, the tools of genetic engineering/gene therapy have steadily improved. Lymphocytes have been attractive targets for these new techniques, for a variety of reasons. The state of the art in engineering T cells, for example, carry designed T cell receptors, has advanced dramatically. This has created new opportunities to design the engineered receptors rationally and intelligently; and to expand the base of such activity to B cells as well.

Described herein are methods for combining a plurality of biological methodologies in new ways to improve the treatment of autoimmune diseases and associated disorders. In some embodiments, the described methods of treatment incorporate a procedure that results in an in situ impact on the autoreactive T cells targeting an individual's tissue (such as BCG vaccination). In some embodiments, the methods described herein comprise techniques for analyzing an individual's repertoire of lymphocyte receptors. Also described herein are methods that involve extracting lymphocytes, manipulating them ex vivo for therapeutic purposes, and then returning those lymphocytes to the individual to induce a therapeutic result.

One embodiment of the present invention describes a technique by which a procedure may be employed to elicit a specific tolerance induction event. This STI event may be analyzed in detail by receptor repertoire sequencing of lymphocytes. The analysis may be expected to reveal the receptor sequences of lymphocyte clonotypes that are specific to the autoimmune disease. These sequences may be used to genetically engineer regulatory lymphocytes that have the same or similar autoreactive T cell specificity but which may be manipulated ex vivo to enhance their anti-autoreactive T cell efficacy when returned to the body as an immunotherapy.

In one aspect, the invention combines an in situ treatment that induces a specific tolerance induction event, such as vaccination or administration of immune system inhibitors, a series of one or more measurements of the T and/or B cell receptor repertoire, an analysis of the T and/or B cell repertoire measurements in order to identify specific T and/or B cell receptor sequences expressed in specific T and/or B cell clonotypes that surge in abundance subsequent to the specific tolerance induction event, and T and/or B cell gene therapy techniques to employ the identified TCR and/or BCR sequences for therapeutic purposes. Autologous T and/or B cells can be engineered using well-established protocols set forth elsewhere herein and well-known to a skilled artisan to express the identified TCR and/or BCR and/or CAR therein; the autologous T and/or B cells can be expanded in culture, again using protocols that are tried and true in the art; and the engineered autologous T and/or B cells once expanded can then be infused or otherwise administered into the patient whose cells they were in the first place.

Further to the discussion above relating to sources of lymphocyte-containing samples, in some embodiments, lymph material or autoimmune target tissue or bone marrow is removed or drawn from the patient even though a sample of peripheral blood would suffice. We know that lymphocytes concentrating at the site of autoimmune disease, in those autoimmune diseases that include such concentrating of lymphocytes, that is, will provide information faster than the lesser concentrations of relevant lymphocytes found in the peripheral blood. For example, biopsy material from diseased bowel, synovial fluid from an affected joint, and cerebrospinal fluid (“CSF”) from autoimmune brain disease can each be expected to include a heightened concentration of lymphocytes reacting to disease processes so located. For example, heightened levels of tumor necrosis factor (“TNF”) can be observed in CSF as opposed to blood in both Alzheimer's disease and mild cognitive impairment; moreover, published data shows correlation of disease progression to increasing TNF in the CSF. At the same time, lack of increasing TNF levels in peripheral blood has been shown in patients whose CSF showed a 25-fold excess of TNF. Tarkowski et al., J. CLIN. IMMUNOL. 19(4):223-30 (1999).

The present method includes protocols for testing the autologous or allogeneic engineered T cells prior to administering them to the patient. These autologous or allogeneic engineered T cells include transgenic T cell receptors (TCRs) or chimeric antigen receptors (CARs) that are either derived from or consistent with safe use in the particular patient; and, prior to administration thereof, are shown to be efficacious in vitro in mounting a therapeutic immune response to the presence of autoimmune target tissue or lysate thereof.

The positive effects of the present invention are observed in vivo upon administration to the patient by noting clinical endpoints such as reduction in inflammatory activity at or about autoimmune target tissue. See, e.g., Eshhar et al., GASTROENTEROLOGY 134:2014-2024 (2008). As described by Eshhar et al., T_{reg} cells function in colitis to suppress autoreactivity of the immune system; and, by extension, in other autoimmune diseases as well.

Another embodiment provides a method wherein autologous engineered T cells, having transgenic T cell receptors (TCRs) or chimeric antigen receptors (CARs), are shown to be efficacious in vivo in affecting surrogate endpoints such as longitudinal measurements of biomarker levels. Suitable biomarkers for such measurements include c peptide and/or serum insulin with respect to diabetes and CRP with respect to inflammatory processes.

Another embodiment provides a method where the STI event is caused by or associated with a medical procedure, wherein the medical procedure comprises one or more surgical procedures, nonsurgical interventions or pharmaceutical treatments. For example, one embodiment for treatment of psoriasis includes PUVA treatment.

Another embodiment of the present invention includes an onset of disease or injury or an intervention/treatment thereof, where a change in the population or repertoire of lymphocytes is observed and used to generate immunomodulating reagents. The immunomodulating reagent, in one such embodiment, can be one or more cytokines administered proximately to the onset of disease or injury for a general immunomodulatory effect at a time when the immune system is usefully down regulated to avoid or reduce inflammation to the autoimmune target tissue or injury. In a second embodiment, the immunomodulating reagent can be lymphocytes having a TCR, BCR, or CAR consistent with one of the identified newly abundant clonotypes identified in accordance with the present invention.

Another embodiment provides a method wherein the sequences of two chains comprising a lymphocyte receptor (e.g., alpha and beta TCR, gamma and delta TCR, or heavy chain and light chain BCR) are paired. The chain pairing is carried out in silico by computer methods, or by use of immunology gene alignment software, or by using VDJ antibodies in accordance with knowledge generally known in the art, or by using multi-well sequencing as known in the art, or by manual means for assessing the DNA sequences of expected chain pairs as know in the art; wherein, in one embodiment, the software is selected from IMGT, JOINSOLVER, VDJSolver, SoDA, iHMMune-align, or other similar software tools.

Another embodiment provides a method comprising obtaining antibodies for the identified VDJ gene segments and using the antibodies to purify a subset of cells which express that gene segment in their (surface) receptors (e.g., using FACS, or immunomagnetic selection with microbeads).

Another embodiment provides a method further comprising sequencing a subset of cells which have been purified for the desired VDJ gene segments.

Another embodiment provides a method further comprising genetic engineering of autologous T cells to display the TCR of the induced clonotype(s), or to display a functional TCR, or to display an alternative type of receptor, such as a B cell receptor. In one further embodiment, the engineered autologous T cells are incubated with autoimmune target tissue and/or lysate thereof prior to administering same to the patient by infusion or other in vivo treatment. One or more criteria are commonly measured during the incubation, such as cytokine concentration, cell proliferation, and the like. In some embodiments, the effects of various adjuvants are quantified. After administration of the engineered autologous T cells, overall survival, progression free survival, tumor regression, and other clinical end points are measured and recorded. In another embodiment, the clinical benefit of treatment with engineered autologous T cells is measured in terms of surrogate endpoints such as longitudinal measurements of biomarker levels or circulating tumor cells.

In one embodiment, lymphocytes bearing particular V and/or J segments are filtered and isolated in a filtration and purification step and incubated for autologic or allogenic cell transfer therapy without genetically engineering lymphocytes to express specific receptors on the cell surface. For this embodiment, spectratyping is carried out on one or more of T cell receptor alpha chains, beta chains, gamma chains, or delta chains; or, in the same fashion, B cell chains can be the subject of the spectratyping. As a result of spectratyping, a change in amount or composition of one or more V or J segments in one or more chains is identified if it exists in the differences between the before and after STI event blood samples.

For example, following a medical procedure that induces a specific tolerance induction event, such as, without limitation intended, application of PUVA for treatment of psoriasis, a qualitative or quantitative increase in amount and oligoclonality may be seen in segments TRAV8-2, TRAJ4, TRBV17, and/or TRBJ2-5 (as defined in the book THE T CELL RECEPTOR FACTS BOOK, by Marie-Paule Lefranc and Gerard Lefranc). Consequently, using appropriate commercial antibodies which are specific to the V and/or J segments identified, cells bearing receptors containing these segments are isolated by a standard and known method for sorting cells, such as fluorescence activated cell sorting (FACS), magnetic beads (Life Technologies dynabeads), or related methods (pluriSelect GmbH cell isolation technologies, to name another).

These autologous cells, bearing desired V and/or J segments, are a “subpopulation” or “subcompartment” of the overall T cell or B cell repertoire. The isolated subcompartment or subpopulation of T cells may then be manipulated or activated in vivo using a number of methods which will be known to one skilled in the art, including cytokines, anti-CD3/anti-CD28 antibodies (Catalog ##111-31D, 111-32D, 111-61D; Life Technologies Corporation, Grand Island, N.Y.), etc. These methods are used, for example, in T cell proliferation assays.

Finally, the relevant subcompartment or subpopulation of cells, possibly having been expanded, activated, or manipulated in vitro, may be therapeutically reinfused. Their persistence and status may also be monitored post-infusion, with subsequent blood draws, as will be known to one skilled in the art. It is notable that this particular embodiment is relatively rapid and cost-effective, since spectratyping may be used instead of sequencing, and isolation using V and J specific antibodies may be used instead of genetic engineering. Further, by avoiding genetic engineering, this embodiment avoids problems with unwanted chain pairings.

In another embodiment, both spectratyping and sequencing may be done, where spectratyping reveals the V and J segments of interest, and sequencing is done using only primers which correspond to the V and J segments of interest. This approach greatly reduces the amount of sequencing necessary, and, as well, provides the opportunity to utilize more rapid sequencing technologies.

In another embodiment a “panning experiment” is carried out after identifying expanded receptors before and after sequencing in which many copies of the identified receptor are incubated with a combinatorial library of possible epitopes. Once the corresponding epitope is identified, tetramers loaded with the identified epitope are used to isolate lymphocytes from the patient's own cells which are specific to that epitope. These cells can then be expanded ex vivo, manipulated, and then therapeutically infused. One skilled in the art is familiar with the procedure (Li Pira et al., J. BIOMED. BIOTECHNOL. 2010:325720 (2010); Sung et al., J. COMPUT. BIOL. 93:527-39 (2002).

One embodiment provides a method for identifying the DNA or RNA sequences of lymphocyte receptors expressed by lymphocytes that are present in increased numbers in a particular patient after a medical procedure, the method comprising: (i) drawing blood or collecting a sample of lymphocytes at one or more times from a patient prior to an STI event; (ii) carrying out or observing an STI event; (iii) drawing blood or collecting a sample of lymphocytes from the patient at one or more times following the STI event; (iv) purifying a lymphocyte subpopulation from the blood or tissue samples, isolating DNA, amplifying (if necessary) and sequencing the genetic loci of lymphocyte receptors; and (v) identifying lymphocytes and/or lymphocyte receptor sequences that have expanded in number following the medical procedure.

A first aspect of the present invention relates to a method of treatment of a patient who suffers from an autoimmune disease, wherein the patient undergoes or experiences a specific tolerance induction (“STI”) event, of which the method includes: (a) optionally collecting a first lymphocyte-containing sample from the patient prior to the STI event (“pre-STI event sample”); (b) detecting an STI event or performing a procedure that correlates in time to an STI event; (c) collecting a second lymphocyte-containing sample from the patient after the STI event (“post-STI event sample”); (d) preparing lymphocytes from the pre-STI event sample or the post-STI event sample; (e) preparing and sequencing DNA or cDNA derived from the prepared lymphocytes; (f) identifying sequences of prevalent T cell receptors (“TCRs”) or B cell receptors (“BCRs”) (collectively, “prevalent receptor sequences”) derived from the post-STI event sample; (g) selecting a regulatory lymphocyte that carries at least one of the prevalent receptor sequences or a sequence that is at least 85% related to one of the prevalent receptor sequences, which selected regulatory lymphocyte (i) expresses one or more prevalent receptor sequences or (ii) is generated from an autologous or allogeneic naïve lymphocyte, which naïve lymphocyte is engineered and induced to become a regulatory lymphocyte that expresses at least one prevalent receptor sequence; (h) culturing the selected regulatory lymphocyte, thereby generating daughter cells of said regulatory lymphocyte; and (i) administering said daughter cells to said patient. If the first lymphocyte-containing sample was collected from the patient, then step (f) hereof further comprises identifying one or more prevalent receptor sequences derived from the post-STI event sample relative to TCRs or BCRs derived from the pre-STI event sample. The prepared lymphocytes of this aspect of the invention include cells displaying various cell surface molecules (e.g., cluster of differentiation or cluster of designation molecules) which are used for immunophenotyping of lymphocytes.

One embodiment of this aspect provides a method wherein the STI event comprises one or more nonsurgical interventions or pharmaceutical treatments; wherein the STI event leads to an immunogenic response.

In another embodiment of this first aspect, step (a) is not optional. In that event, the inventive method further includes the step of identifying clonotypes from the post-STI event sample that are not identified among clonotypes from the pre-STI event sample or that have an expansion frequency of 0.5% or greater relative to the clonotypes from the pre-STI event sample. The step of identifying clonotypes, in one embodiment, includes spectratype analysis or sequencing DNA or cDNA or both.

Another embodiment provides a method further comprising selecting a clonotype if it is highly expanded both before and after an STI event, but has a frequency that increases from before to after the STI event, wherein the increase is statistically significant using an appropriate multiple hypothesis testing statistical method to limit the false discovery rate stringently.

When spectratype analysis is an included step, then the inventive method further includes the steps of: (a) identifying one or more V or J segments among the prevalent receptor sequences; and (b) selecting a regulatory lymphocyte bearing the identified V or J segment(s).

The selected regulatory lymphocyte identified in the context of an embodiment of this aspect of the invention is a regulatory T cell (“T_{reg}”), wherein the selected regulatory lymphocyte includes CD4⁺, CD25⁺, or FoxP3⁺ and one or more genetic markers selected from the group consisting of CD25^{Hi}, CD197+, CD194+, CD103⁺, Folr4⁺/FR4⁺, Nrp1⁺, HLA-DR⁺, CD279⁺, CD134⁺, CD137⁺, LAP⁺, GITR⁺, CD152⁺, CD45RA⁺, and CD127^{low}.

In another embodiment of this aspect of the invention, the selected regulatory lymphocyte is a regulatory B cell (“B_{reg}”), wherein the selected regulatory lymphocyte includes CD5⁺ and CD1d⁺ and one or more genetic markers selected from the group consisting of TIM-1⁺, IL-10⁺, CD21^{hi}, CD23⁺, CD24⁺, CD27⁺, and CD38⁺.

In another aspect of the present invention, the selected regulatory lymphocytes are cultured in vitro. Such culturing of the selected regulatory lymphocytes is also referred to as ex vivo culture, resulting in expansion of the number of cells due to increasing numbers of daughter cells. In one embodiment, the daughter cells are administered in combination with a cytokine. Preferably, the cytokine is interleuken 10 (“IL-10”) or transforming growth factor beta (“TGF-β”).

In one embodiment of the present invention, a biological drug or small molecule drug that suppresses the patient's immune response is administered. The biological drug or small molecule drug is preferably selected from the group consisting of a tumor necrosis factor alpha (“TNF-α”) blocker, etanercept, an anti-T cell antibody, azathioprine, cyclophosphamide, cyclosporine, methotrexate, mycophenolate, sirolimus, tacrolimus, and a cytokine. In one embodiment, the biological drug or small molecule administered is at least two cytokines; in another embodiment, the at least two cytokines include interleuken 10 (“IL-10”) and the transforming growth factor beta (“TGF-β”). In one embodiment, the biological drug or small molecule is administered proximate to the time of the STI event.

In another embodiment, the inventive method includes enhancing the efficacy of the selected regulatory lymphocyte in vitro. Such enhancement is accomplished by employing genetic engineering protocols referred to elsewhere herein to cause autologous or allogeneic lymphocytes to express the receptor(s) of the induced clonotype(s); or, more simply, expanding an identified regulatory lymphocyte and re-infusing same to the patient without more. In one embodiment, the method includes engineering a lymphocyte to display a functional TCR identified from the induced clonotype(s) or an alternative type of receptor such as a B cell receptor or a chimeric antigen receptor (CAR). Employing steps to enhance the efficacy of the selected regulatory lymphocyte can occur with or without administration of the aforementioned biological drug or small molecule.

Another embodiment provides a method wherein the analysis of lymphocyte receptor repertoire sequences, from patient samples obtained both before and after an STI event, enables the production of autologous genetically engineered T cells having chimeric antigen receptors which are specific to the patient's autoimmune target tissue.

Another embodiment provides a method wherein autologous engineered T cells, having transgenic T cell receptors (TCRs), B cell receptors (BCRs) or chimeric antigen receptors (CARs), are shown to be efficacious in vivo in affecting clinical endpoints such as reduction of inflammation directed at autoimmune target tissue or improved self-regulation of serum sugar levels with respect to diabetes, offered as two non-limiting examples.

Another embodiment provides a method wherein autologous engineered T cells, having transgenic T cell receptors (TCRs) or chimeric antigen receptors (CARs), are shown to be efficacious in vivo in affecting surrogate endpoints such as longitudinal measurements of biomarker levels or autoreactive lymphatic cells.

Another embodiment provides a method further comprising an in vitro assay for assessing the effectiveness of the autologous engineered lymphocytes.

Another embodiment provides a method wherein, the ex vivo expanded lymphocytes are incubated with autoimmune target tissue and/or lysate.

Another embodiment provides a method wherein one or more effects are measured during the incubation such as cytokine concentration, cell proliferation, and the like. In some embodiments, the effects of various adjuvants are quantified.

A second aspect of the present invention relates to a method for identifying regulatory lymphocyte clonotypes that have expanded in a patient following a specific tolerance induction (“STI”) event or a procedure that correlates in time to an STI event, comprising the steps of: (a) optionally collecting a first lymphocyte-containing sample from the patient, wherein the first lymphocyte-containing sample is collected prior to the STI event; (b) recording the STI event or performing the procedure on the patient; (c) collecting a second lymphocyte-containing sample from the patient, wherein the second lymphocyte-containing sample is collected subsequent to the STI event; (d) spectratyping lymphocytes from the first lymphocyte-containing sample, if said first lymphocyte-containing sample was collected, and from the second lymphocyte-containing sample; and (e) identifying regulatory lymphocyte clonotypes that have expanded following the STI event. This second aspect can further include the steps of: (a) purifying lymphocytes from the first lymphocyte-containing sample; and (b) purifying lymphocytes from the second lymphocyte-containing sample.

A third aspect of the present invention relates to a therapeutic composition for treating a patient afflicted with an autoimmune disease, which composition includes cultured regulatory lymphocytes, wherein the cultured regulatory lymphocytes are substantially enriched for those that express up to six different TCRs or BCRs; in other embodiments thereof, the cultured regulatory lymphocytes are substantially enriched for those that express up to about six different TCRs or BCRs; or up to about five different TCRs or BCRs; or up to about four different TCRs or BCRs; or up to about three different TCRs or BCRs; or up to about two different TCRs or BCRs. In yet another embodiment, the cultured regulatory lymphocytes are substantially enriched for those that express six different TCRs or BCRs; or five different TCRs or BCRs; or four different TCRs or BCRs; or three different TCRs or BCRs; or two different TCRs or BCRs; or one TCR or BCR. For this aspect of the invention, the TCRs or BCRs are identified from analysis of a lymphocyte-containing sample removed from the patient after a specific tolerance induction (“STI”) event.

The TCRs or BCRs so included in the therapeutic composition are specific to the patient to be treated, and are identified from analysis of a lymphocyte-containing sample removed from the patient after a specific tolerance induction (“STI”) event, as disclosed herein above.

The therapeutic composition, in some embodiments, further includes a cytokine; and in some other embodiments, further includes two different cytokines.

The cultured regulatory lymphocytes of the therapeutic composition, subsequent to administration into the patient, specifically suppress one or more immune system functions involved in the patient's autoimmune disease.

In some embodiments of the present invention, the cultured regulatory lymphocytes are derived from an autologous lymphocyte isolated from the patient or a lymphocyte that is allogeneic with respect to the patient. The cultured regulatory lymphocytes can be expanded ex vivo using standard methods well known in the art.

A beneficial effect can easily be assessed by one of ordinary skill in the art and/or by a trained clinician who is treating the patient. The term, “disease” refers to any deviation from the normal health of a mammal and includes a state when disease symptoms are present, as well as conditions in which a deviation (e.g., infection, inflammation, etc.) has occurred, but clinically-notable symptoms are not yet manifested.

As will be further described below, the present invention is predicated on close observation of lymphatic cell populations of the patient before and after a specific tolerance induction event that is caused either by a natural but deleterious process, such as a flaring of an autoimmune disease, or trauma involving cell death, as occurs in an infarct of brain or heart tissue, or in a head or spinal cord injury. Inflammatory processes that proceed after infarct events (meaning that the organ said to experience the infarct consequently includes dead tissue) associated with heart or brain, for example, can be controlled or reversed using the methods and materials set forth herein below. In particular, the present invention can be used to treat the inflammatory processes that occur subsequent to brain infarction, coronary artery disease, myocardial infarction, periodontal disease, brain trauma, spinal cord trauma, and the like.

The treatment design of the present invention is directed at causing down-regulation of the immune system. Unfortunately, the regulatory lymphatic cells that are self-selected to retard overzealous immune activity may be ineffective for any number of reasons, including there being too few, or having cell receptors that have insufficient affinity for the immune cells that are causing the harmful inflammatory effect. Procedures set forth herein below address such scenarios and provide methods and materials to introduce to the patient a product that upticks the patient's own immune system strategy for down regulating the body's own autoreactive cells, when considering self tissues or organs, or graft-reactive cells, when considering materials purposely introduced into a patient.

The immune system is the body's police, seeking to maintain or restore inner peace by identifying and destroying carriers of foreign antigens as well as down-regulating certain actors from continuing seek-and-destroy missions once the foreign antigens, for example, have been rendered harmless and from misguided seek-and-destroy missions directed at self antigens. The inventive methods set forth herein collect information from the police (i.e., the body's immune system as observed in one's peripheral lymphocytes by one skilled in the art and knowledgeable of the present invention) and use that information to (a) identify regulatory lymphocyte clonotypes that can be usefully employed to ameliorate the disease or injury that gave rise to the identified clonotypes, and (b) generate autologous regulatory lymphocytes of the identified clonotypes for infusion into the patient for waging an immunotherapeutic attack to a clinically observable level.

The present invention uses current understandings of the immune system to identify clonotypes of regulatory lymphatic cells that may be employed for selecting suitable regulatory lymphocytes from the patient's blood that may then be expanded in culture and then returned to the patient's bloodstream to further the immunotherapeutic attack that those cells had started but could not complete for lack of numbers.

The description and examples presented herein below set forth methods and materials learned and designed based on close observation of the body's police force, the immune system. Using the descriptive capabilities of contemporary molecular biology, we have made use of the phenomenal contemporary capability to sequence chromosomal DNA from lymphocytes, irrespective whether first purified or not. In particular, the present invention, in one embodiment, utilizes our ability to focus on the gene responsible for the T cell receptor (“TCR”) or the B cell receptor (“BCR”), especially its V and J regions that are known to those skilled in the art to recombine in myriad variations. After recombining those regions of the lymphocytes' genomes, the recombined TCR/BCR genes reside respectively within the genome of clonally related lymphocytes, hence that term “clonotypes” already used above. Using well-described primers that target the V and J regions, one can literally sequence those regions and so determine what specific TCRs or BCRs are present in the population of lymphocytes. Furthermore, the sequence data also provides quantitative data for assessing the relative abundance of lymphocytes having particular TCR or BCR sequences in a blood sample, which in turn defines for us the most abundant clonotypes in the blood sample.

The invention being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

### EXAMPLES

### Example 1

Cells employed for treating a patient afflicted with an autoimmune disease using methods and materials of the present invention are designed specifically for each patient based on a clonotype analysis that compares the receptors found on populations of lymphocytes obtained from the patient before and after s/he experiences a specific tolerance induction (“STI”) event. Example 1 illustrates alternative protocols for such clonotype analysis.

The populations of lymphocytes to be compared are collected from the patient prior to and five to 10 days after the STI event, which are respectively referred to herein as the “first lymphocyte sample” and the “second lymphocyte sample”. For STI events that are scheduled in advance, as in a scheduled surgery or vaccination or other treatment ordered by the patient's physician, obtaining the two samples of lymphocytes by venipuncture or other standard protocols are readily scheduled as well.

However, the present invention is also effective for autoimmune patients who experience unscheduled STI events, such as an infarct or other injury or a flare of symptoms that occur by natural processes or accident if the lymphocyte samplings are completed reliably in accordance with the timing set forth here. With regard to a first lymphocyte sample (i.e., a pre-STI event lymphocyte sample), the physician orders collection of the first lymphocyte sample as soon as possible after the STI event begins; preferably, the medical records of the patient will indicate that a prior first lymphocyte sample was collected and analyzed and/or stored for later analysis using procedures well-known in the art. Having an earlier lymphocyte sample collected at a time at least a month after a STI event had resolved is a useful tool for confirming the reliability of the sample collected after the STI event commences; the spectrum of receptor sequences would be similar between the two “pre-STI event” lymphocyte samples, and neither would include the heightened level of receptor as found in the post-STI event lymphocyte sample (i.e., the second lymphocyte sample) collected about 5-10 days after that event. This analysis of pre- and post-STI lymphocyte samples is required in order to determine the one or more lymphocyte clonotypes (defined by the included receptors) that achieves increased concentration in the fluid or tissue from which the lymphocytes are sourced, as appropriate. The clonotype information is then used to select or generate cells for expansion sufficient for treatment of the patient.

Irrespective of the method of clonotype analysis used, samples are obtained that include lymphocytes, such as peripheral blood, lymphatic tissue, and/or tissue at site of inflammation—at least one prior to the specific tolerance induction (“STI”) event and at least one after the STI event (e.g., 5, 6, 7, 8, 9, or 10 days afterwards). The effect of the STI event will begin waning at about two weeks after, so it is preferred to collect the post-STI event at least by the two week mark. Where possible, we obtain additional samples at earlier or later time points, respectively.

Lymphocyte samples are collected using procedures well-known to the art, such as collection of peripheral blood by venipuncture (e.g., a 10 ml whole blood taken per sample), or apheresis, more particularly leukopheresis, or collection of lymph glands by biopsy procedures. Additionally, samples of affected tissue that has been infiltrated by lymphocytes is an excellent source, and can be accessed using reasonably patient-friendly methods involving ultrasound for locating the tissue, if necessary, and a needle.

With respect to samples of peripheral blood, we isolate mononuclear cells (i.e., the lymphocytes) therefrom by flotation on a Ficoll-sodium metrizoate gradient of specific density, centrifugation, and collection of the lymphocytes from the plasma-Ficoll interface. Multiple lymphocyte subsets are enriched from freshly isolated peripheral blood mononuclear cells by immunomagnetic selection with microbeads (e.g., Miltenyi CD8+ kit) or other separation methods such as fluorescence-activated cell sorting (“FACS”). We isolate one or more of the following subsets: B cells, CD8⁺ T cells, CD4⁺ T cells, CD4 Th1 cells, CD4 Th2 cells, CD4 Th17 cells, T_{reg} cells (nTreg, iTreg, Th3, Tr1), NKT cells, gamma-delta T cells, and dendritic cells. Depending on blood volumes, we separate into subsets based on naïve, effector, or memory subsets, as known in the art.

An alternative method for isolating T_{reg} cells following a protocol of U.S. Application 2011/0300119 follows: Cell leukapheresis products containing approximately 5×10⁹ cells are obtained from the autoimmune patient. Peripheral blood mononuclear cells (PBMCs) are obtained from 5 to 10 ml blood from patients. PBMCs are prepared over Ficoll-Paque Plus gradient centrifugation (GE Healthcare, Little Chalfont, United Kingdom). For FACS, CD4⁺ cells are enriched over the AutoMACS Pro Separator by positive selection with human CD4⁺ microbeads (Miltenyi Biotec, Auburn, Calif.). The cells are labeled with CD4 FITC, CD25 PE, CD45RA PE-Cy5.5 (all Invitrogen, Carlsbad, Calif.) and CD127 Alexa Fluor 647 (BD Biosciences, San Jose, Calif.). The FACSVantage DiVa or FACSAria flow cytometer is used to sort T_{reg} cells by gating on the top 2% CD25′” and non-T_{reg} cells by gating on CD4⁺ CD25 CD127⁺ CD45RA⁺ cells. For magnetic bead purification based on CD25, the Miltenyi CD4⁺ CD25⁺ Regulatory T Cell Isolation Kit is used with a modified manufacturer's protocol. In brief, all non-CD4⁺ cells are depleted over the AutoMACS Pro with a cocktail of biotin-conjugated monoclonal antibodies (“mAbs”) against CD8, CD14, CD16, CD19, CD36, CD56, CD123, TCRγ/δ, and CD235a. The unlabeled CD4⁺ T cells are incubated with CD25 microbeads (5 μl/10⁷ CD4⁺) and positively selected over the AutoMACS Pro with Posseld2 program. For magnetic bead purification based on CD25 and CD127, the Miltenyi CD4⁺ CD25⁺ CD127^{dim/-} Regulatory T Cell Isolation Kit is used with a similar protocol. For the one-step method of CD25⁺ cell purification, total PBMCs are incubated with CD25 microbeads (2 μU10′ cells) for 20 minutes at 4° C. and positively selected over the AutoMACS Pro with Posseld2 program. For magnetic bead sorting of in vitro-expanded LAP⁺, CD121a⁺, or CD121b⁺ cells, the cells are stained with anti-LAP⁺ phycoerythrin (“PE”) or anti-CD121a PE followed by anti-PE microbeads or anti-CD121b biotin followed by antibiotin microbeads (both Miltenyi Biotec) then positively selected over the AutoMACS Pro with Possels and repeated with Possel program. PE is a fluorescent dye.

Antibodies conjugated to PE are as follows: CD73, CD120b, CD127, CD134, CD137, CD278 (from BD Biosciences); CD39, CD101 (from eBioscience, San Diego, Calif.); and GITR (from Miltenyi Biotec). For staining of CD121a, CD121b, and LAP, anti-LAP PE or anti-CD121a PE (both R&D Systems, Minneapolis, Minn.) and anti-CD121b biotin followed by secondary staining with streptavidin PE or APC (BD Biosciences) were used. For intracellular staining of FOXP3, the cells are fixed and permeabilized with a Fixation/Permeabilization kit and stained with anti-FOXP3 mAb Alexa Fluor 488 or 647 clone 236A/E7 (eBioscience). All cells are cultured in complete media consisting of RPMI 1640 supplemented with 5% heat-inactivated autologous serum, penicillin (100 U/ml), streptomycin (100 μg/ml), 2 mM L-glutamine, 10 mM HEPES, 0.1 mM nonessential amino acids, 1 mM sodium pyruvate and 50 μM 2-mercaptoethanol.

Other protocols are available from the published literature for collecting regulatory lymphocytes, such as: U.S. Patent Applications 2009/0208471, 2009/0136470, 2010/0068193, 2010/0111916, 2010/0111982, 2010/0129340, 2010/0189728, 2011/0097334, and 2011/0123502, and U.S. Pat. No. 7,695,713; each of which are incorporated herein by reference.

Once the lymphocyte cells have been purified as a single set or as any of the possible subsets, they are subjected to clonotype analysis by one or both of the methods set forth herein below.

For either of the methods, the definition of an induced clonotype is a clonotype whose change in frequency between a prior STI event sample to a post-STI event sample is above a defined threshold. We define the threshold as 0.5%, a conservative frequency which is used to define a highly expanded clone, and which is supported by vendor reproducibility data (with respect to frequencies of receptor sequences therein). The analysis is also repeated with higher and lower thresholds (down to 0.1%). We rank and characterize clonotypes as weakly or strongly emergent based on their percentage increase in frequency. For example, a clonotype which had a frequency of 0.1% prior to the STI event and 0.9% afterward has an increase in frequency of 0.8%; as the difference is greater than the minimum increase of 0.5%, we characterize this clonotype as induced.

With multiple patient samples, we analyze the data for the presence of “public clonotypes”, which are defined as identical (or similar) sequences which arise in parallel in multiple patients. Public clonotypes represent a valuable grouping/segmentation of the patients. We analyze the clonotype groupings, if any, in relation to clinical outcomes. For in vivo and in vitro treatments, described below, we also analyze whether particular public clonotypes have favorable or unfavorable responses to in vitro or in vivo treatment.

DNA Sequencing Method of Clonotype Analysis: We extract total genomic DNA from sorted cells using the QIAamp DNA Blood Mini kit (Qiagen) or similar kits, or commercial services providing DNA extraction or isolation. We prepare and ship DNA as per sequencing vendor instructions: at a concentration of approximately 50 ng/μl, with an A_{260/280} ratio of at least 1.8, and shipped with dry ice using a vendor-supplied shipping container. Note: It is equally valid to conduct sequencing based on RNA species present as well. Note that this procedure results in pooled genomic DNA, for which an additional analysis step for pairing receptor chains is required. At a first level of analysis, we identify receptor sequences of cell clonotypes that are induced or expanded based on the definitions provided above. We further characterize the lymphocyte receptors knowing that each consists of two chains that are paired in situ. For example, in T cells, a receptor may consist of an alpha and a beta chain; a different receptor may consist of a gamma and a delta chain. In B cells, the two chains of the relevant receptor are the heavy chain and the light chain. In the following explanation, for convenience, we refer to alpha and beta chains, but a similar strategy is used for pairing heavy chains and light chains, or gamma chains and delta chains; and in all cases understand that the pooled sequencing as used results in a list of induced alpha chains, and a list of induced beta chains (or heavy chains and light chains, etc), and we now pair the chains.

Alternatively, we extract total RNA as set forth herein above at Paragraph [0065].

In order to pair the chains, we benefit from the fact that these chains are made in vivo via VDJ recombination. Furthermore, V and J gene segment specific antibodies are commercially available.

Therefore, we start with one chain—for example, the beta chain. We identify an induced clonotype, and from its sequence, we identify its V and J gene segments, as follows.

We can annotate the induced clonotype's gene segments from its sequence using specialized, publicly available immunology gene alignment software from IMGT (International IMmunoGeneTics information system, www.imgt.org, V-Quest, JunctionAnalysis, etc.), JOINSOLVER, VDJSolver, SoDA, iHMMune-align, or other sources that have similar tools.

We obtain antibodies for the identified segments. We use the antibodies to purify a subset of cells which express that gene segment in their (surface) receptors (e.g., using FACS, or immunomagnetic selection with microbeads). Finally, using this subset of cells which have been purified for the desired gene segments, we sequence again, as described above, or in a less expensive, more low-throughput manner. If necessary, we sequence this subset more deeply (i.e., at a higher resolution) than previously. In this new data set, there will be far fewer induced clonotypes, greatly easing the task of chain pairing. Depending on the gene segments, there may be only one induced alpha chain and one induced beta chain for example.

Alternatively, (e.g., if the above method is inconclusive), we can isolate our gene segment purified cells (or even unpurified cells) from a microwell plate, where each microwell has a very low number of cells. We amplify and sequence the receptors of cells in each well individually, which provides another means to pair the chains of interest by sequencing on a single cell basis (or single clonotype basis, if we treat the cells (e.g., with adjuvants) to proliferate in the microwells prior to sequencing them).

An alternative sequencing method reduces the need for the step of pairing receptor chains, namely high throughput microdroplet-based single cell analysis (such as that offered by RainDance, Inc.); vendor instructions are followed to prepare for single cell sequencing. In that event, the chain pairing is known without further effort.

Spectratype analysis for identifying clonotypes is also available for assessing the induced clonotype. In spectratype analysis, one uses antibodies that identify defined epitopes associated with the receptors; the protocol is described in detail herein above.

### Example 2

Once the information is available from the clonotype analysis in accordance with protocols set forth in Example 1, wherefrom we know the receptor sequences of the one or more induced clonotypes that arise subsequent to an STI event, we construct an autologous engineered T cell or B cell or a regulatory T cell or a regulatory B cell or expand numbers of cells of the identified clonotype using the induced clonotype information that we identify as described above. This engineered cell or expanded population of the identified clonotype is the basis of an immunotherapy which we pre-test in vitro for safety parameters and, if the pre-test passes, we use therapeutically in vivo for the patient from whom the cells were originally sourced. Example 2 illustrates protocols according to one embodiment of the present invention for generating autologous cells that may or may not have been engineered to express exogenous DNA, as noted below.

Using the information derived in Example 1 of the paired alpha/beta, gamma/delta, or heavy/light chains of the induced clonotypes, we set forth to create an expanded population of autologous lymphocytes that express a receptor corresponding to that associated with the induced clonotype. This receptor may be a T cell receptor on a T cell surface, a B cell receptor on a B cell, or a chimeric TCR or BCR that may be expressed on either a T cell or a B cell. The chimeric receptor may be comprised of a single chain variable fragment (scFv), as one example; or include costimulatory endodomains. Generating a recombinant lymphocyte that contains the TCR, BCR, or CAR of interest can be accomplished using protocols that are well known in the art, and described in various places, including Nishimura and Rosen, United States Patent Application US20100172888 (2010).

The simplest approach is to isolate additional lymphocytes from the patient that already express the identified receptor, select the specific cells identified by the clonotype analysis of Example 1, and culture them under conditions that stimulate cell divisions and thereby generate an expanded population of the very cells that were induced by the STI event. We grow the selected cells in good manufacturing practice (GMP)-grade CellGro medium (CellGenix, Breisgau, Germany) supplemented with 10% autologous serum (collected from the patient via apheresis and processed using standard protocols known to the art), and interleukin-2 (“IL-2”; 1000 units/ml; Proleukin, Chiron, San Diego, Calif.).

The specific lymphocytes are isolated from the leukocyte sample collected from the patient with a GMP-adapted FACS with single-use sample lines (Influx; BD Biosciences, San Jose, Calif.). The single-use sample lines eliminate the risk of cross-contamination between samples of different patients.

In particular, autologous lymphocyte cells are selected from the patient's lymphocyte samples using the GMP-adapted FACS to select cells that are incubated with clinical grade anti-CD3/anti-CD28 beads (Invitrogen, Carlsbad, Calif.) to which antibodies or portions thereof that are specific for the receptor(s) identified in the clonotype analysis are attached. Such antibodies that are specific for a patient's particular lymphocytes are generated de novo. Alternatively, one identifies the specific V and J segments included on receptors of the patient's induced lymphocytes using commercially available anti-V and anti-J antibodies and, once identified, those same antibodies are included on the anti-CD2/anti-CD28 beads for identifying and isolating induced lymphocytes. In yet a further alternative protocol, we can achieve greater clarity by conducting the FACS protocol in sequence, first using the anti-CD2/anti-CD28 beads followed by using a second set of beads to which the aforementioned anti-V and anti-J antibodies are attached. Note: Using the anti-V and anti-J antibodies to select for induced lymphocytes results in a subset of cells that are at least enriched for the induced lymphocytes, but may include other lymphocytes that share the same V and/or J segments.

Once at least 1 billion daughter cells of the cells selected in the FACS protocol are available for transfusion into the patient, the cells are collected, washed in fresh culture medium and then transfused into the patient. All procedures are standard protocols known in the art, using dosage rates between 10 million and 20 million cells per kg body weight of the patient.

An alternative protocol involves engineering a lymphocyte isolated from the patient so that it expresses the identified receptor on its surface. The tools for this process are commercially available and a great deal of literature describes this process. In particular, recently published work with T cells and CARs (chimeric antigen receptors) provides guidance for enhancing the potency of the engineered lymphocyte cells (in particular, third generation CARs). In such CARs, the receptor used is altered from the native receptor identified through the clonotype analysis to a variant that increases the affinity between the engineered cells and the effector cells that are optimally down-regulated.

When engineering a lymphocyte cell which expresses a desired (induced) receptor sequence, we prepare by acquiring a suitable lentiviral or other retroviral vector. A number of commercial vendors can construct customized lentiviral vectors, and a number of kits for lentiviral transduction are available. In the alternative, it may be beneficial to use other kinds of vectors. In summary, we obtain commercially engineered lentiviral particles in which the desired (induced) TCR, BCR or CAR sequences have been introduced. In addition, we acquire a suitable population of lymphocyte cells from the patient via leukapheresis, and maintain them ex vivo.

Following vendor instructions, we then incubate the lymphocyte population with the lentivirus. Cytokines IL-2 or IL-7 are used to facilitate this process at concentrations set forth in vendor instructions.

Another protocol is employed in the alternative, as set forth in Ellis et al., J. VIS. EXP. 62:e3738 (2012), which is incorporated herein by reference.

We confirm the success of the transduction, and the expression of the engineered lymphocyte cells in multiple ways. First, we use VDJ gene segment specific antibodies, as described previously. Second, we sequence the engineered cells, as described previously. We use additional verification methods as appropriate.

Of course, there are numerous available protocols for ex vivo culturing of isolated lymphocytes once identified in order to expand the population suitably for use in an adoptive cell therapy administration. For example, protocols for ex vivo expansion of regulatory lymphocytes can be found in U.S. Patent Applications 2009/0142317, 2009/0162334, 2010/0260781, 2011/0123590, and 2012/0207727; each of which are incorporated herein by reference.

In addition, the literature provides protocols for removing lymphocyte cells from a patient, expanding a portion ex vivo, and reintroducing same to the same patient, as in U.S. Patent Application 2009/0311228, which is incorporated herein by reference. Such protocols are particularly useful when coupled with the novel features disclosed herein regarding methods for identifying the clonotype receptor sequences of interest, expanding cells that include the identified sequences of interest and returning same to the patient.

### Example 3

This Example illustrates one protocol for treatment of psoriasis using the method of the present invention.

Psoriasis is a common autoimmune disease that affects the skin, causing red raised plaques covered with white scale. It is characterized by abnormal keratinocyte differentiation, hyperproliferation of the keratinocyte, and infiltration of inflammatory elements. Psoriasis is an immune-mediated disease which is commonly treated with immunosuppresive drugs.

One established treatment for psoriasis is PUVA therapy, which acronym stands for psoralen irradiated with ultraviolet A (“UVA”), and which is a form of photodynamic therapy. PUVA therapy is known to induce circulating regulatory T cells in patients with psoriasis. See Saito et al., J. DERM. SCI. 53:231-233 (2009); http://www.ncbi.nlm.nih.gov/pubmed/19070466. Accordingly, such treatment creates an STI event usefully employed for the protocols of the present invention.

By sequencing blood samples from before and after the STI event (i.e., administration of PUVA therapy), T cell receptors (TCRs) of induced regulatory T cells are identified (using the protocols set forth in Example 1 above). With knowledge of the PUVA-induced T cell receptor(s), autologous T cells that express the identified TCR(s) are engineered (or collected from the patient), cultured in vitro, and, upon attaining a population of about a billion daughter cells, are reinfused into the patient.

Using this approach reduces activity of the autoreactive T cells that are responsible for psoriasis. Moreover, since long term PUVA treatment is associated with an increased risk of melanoma (Stern et al., NEW ENGLAND J. MED. 336:1041-45 (1997)), treatment with the infused T cells is preferable for both safety and efficacy compared to repeated PUVA treatment.

### Example 4

This Example illustrates two protocols for treatment of Crohn's Disease using the method of the present invention.

Crohn's disease is an autoimmune-based inflammatory bowel disease which is characterized by chronic inflammation of the gastrointestinal tract. The disease often manifests as alternating periods of flare ups and remission. It causes a wide variety of symptoms such as abdominal pain and weight loss, and can lead to complications such as bowel obstruction, fistulas, and strictures, and may require surgery.

Crohn's disease is characterized by excessive numbers of autoreactive T cells in the mucosa of the gut with abnormal proliferation and cytokine production (these are found in “skip lesions”). These T cells are known to circulate systemically, however a preferred source for assessing the population of autoreactive as well as regulatory lymphocytes is to sample lymphocytes found at the site of the skip lesions by biopsying same. Newer biological therapies of immunosuppression, such as administration of infliximab or adalimumab (which are monoclonal antibodies directed against tumor necrosis factor alpha (“TNF-α”)), has helped a proportion of Crohn's patients, but many remain refractory to these treatments and end up requiring surgery.

One approach is to administer to the patient a low level of autologous mesenchymal stromal (“MS”) cells derived from the patient's bone marrow. About 2 million MS cells (per kg body weight) are delivered by intravenous infusion in accordance with standard protocols known to the art and as published for a study entitled “An Australian Study of Mesenchymal Stromal Cells for Crohn's Disease”, ClinicalTrials.gov identifier NCT01090817 (2010) (available online at http://clinicaltrials.gov/ct2/show/NCT01090817). Lymphocyte samples are collected prior to introduction of the MS cells to the patient and seven (7) days thereafter, followed by clonotype analysis of the lymphocyte samples before and after the MS cell administration, in accordance with the protocols set forth in Example 1. As noted above, circulating lymphocytes and/or lymphocytes found at skip lesions can be assessed.

Thereafter, autologous cells that express the lymphocyte receptors so identified are created and grown in accordance with procedures set forth in Example 2 hereof.

Adoptive cell therapy is then conducted wherein a dose of 10 million to 20 million cells per kg body weight is administered by standard transfusion protocol. Administration of the same dose of cells is repeated weekly up to four weeks, after which the patient is observed for an additional four weeks. It is our expectation that the patient's symptoms will have reduced significantly.

A second approach to generating cells usefully employed in a therapy of the present invention for a Crohn's patient involves sequencing DNA from (1) a first lymphocyte sample collected within one to two days after a flare period commences and (2) a second lymphocyte sample collected between five to six days after the flare period commences and one to two days after a flare period subsides. A clonotype analysis is then conducted using the first lymphocyte sample and the second lymphocyte sample in accordance with the protocols set forth in Example 1 above. In so doing, the dominant lymphocyte receptor(s) associated with lymphocytes present in the patient upon response to the flare are identified. With knowledge of the dominant receptor(s) associated with reduction in flare symptoms, regulatory T cells bearing receptor(s) are generated by cloning such sequences into autologous lymphocytes, induced to become regulatory T cells, cultured to generate at least two billion cells, and then about one billion cells are reinfused into the patient during each of at least two treatments separated by at least 7 days, using the protocols of Example 2 hereof.

A Crohn's patient treated with the cells generated in accordance with the first or second approach experiences a reduction in Crohn's disease activity score by 100 points or more at six weeks post start of therapy, using standard protocols for evaluating Crohn's disease patients.

### Example 5

This Example illustrates one protocol for treatment of Alzheimer's disease using the method of the present invention.

Alzheimer's disease (AD) is associated with amyloid-beta peptides, in both soluble oligomeric and insoluble forms. These peptides have been targeted for vaccine-based therapies of AD. Multiple amyloid-beta (“Aβ”) vaccination strategies in animal models of AD have demonstrated a marked reduction in both amyloid burden and neurocognitive deficits. However, initial human clinical trials of an active Aβ vaccine were discontinued due to the development of meningoencephalitis in approximately 6% of the vaccinated AD patients. Accordingly, components of the Aβ peptide remains part of a vaccine to serve as a STI event for AD patients, but a C-terminal truncation of the Aβ fragment is used to avoid T cell activating epitopes located toward the C terminus and thus more likely avoid the ill-effect of meningoencephalitis side effect seen in early trials. Giunta et al., AGEING RESEARCH 2:e5 (2010). Alternative fragments are employed, including Aβ₁₋₄₂ Aβ₁₋₄₂. Id.

A blood sample is collected from an AD patient prior to being administered an Aβ vaccine, which is presented in an amount that is not associated with the aforementioned meningoencephalitis; and then a second blood sample is collected seven days after vaccine injection. The blood samples from before and after the administration of the amyloid beta vaccination are sequenced in accordance with Example 1, analysis of which reveals the dominant receptor (s) induced by the vaccine. Autologous lymphocytes are then engineered to express the dominant receptors, grown in culture, and ultimately infused back into the patient, resulting in down-regulating the Aβ reactive T cell subsets, ameliorating the deleterious response. Giunta et al., Ageing Research 1(1):e5; available online at http://www.pagepress.org/journals/index.php/ar/article/view/1441.

### Example 6

This Example illustrates one protocol for treatment of multiple sclerosis (MS) using the method of the present invention.

Multiple sclerosis is a chronic relapsing autoimmune disease of the central nervous system manifested by loss of motor and sensory function. The cause of the disease is immune-mediated inflammation, demyelination and, eventually, damage to neuronal axon junctions. Zhang et al., J. EXP. MED. 179(3):973-84 (1994). It is generally accepted that MS is mediated mainly by T cells that target self-antigens associated with the MS-patient's own myelin. A protocol developed in accord with the present invention capitalizes on the phenomenon that mycobacteria induce suppression of autoimmunity in the central nervous system, and thus induces a STI event. See Lee et al., J. NEUROIMMUNE PHARMACOL. 5(2):210-9 (2010). Whereas prior art immunotherapeutic approaches to treatment of MS include interventions aimed at down-regulating the various effector immune cells that are involved in the immunological process, none have provided a regime of immunomodulation by down-regulating the disease-causing immune cells by generating and/or enhancing T_{reg} cells. See Karussis, BIODRUGS 27(2):113-48 (2013); available online at http://link.springer.com/article/10.1007%2Fs40259-013-0011-z.

Animal evidence supports the inventive protocol presented here for treatment of MS patients, as follows: Model mice for MS are employed. The mice are certain transgenic mouse strains, namely NOD, B10.PL, or SJL. MS symptoms are induced in individual mice of such strains by subjecting them to an experimental allergic encephalomyelitis (“EAE”) protocol. For example, SJL mice can be induced with proteolipoprotein (“PLP”) 139-151aa or myelin-oligodendrocyte-glycoprotein (MOG) 1-9aa; and B10.PL mice best respond to PLP 43-64 or MOG 87-106aa. Such experimental animals are available commercially from Biomedical Research Models, Inc. of Worcester, Mass.

MS model mice are #! divided up into two groups of five animals each and two groups of control mice of two mice each. Animals of all groups are induced to display MS-like symptoms of neurological dysfunction using one of the agents noted above as appropriate for the particular strain obtained. The experiment is done in duplicate sets and designed to test a method of therapy, as follows:

A first sample of blood is collected from each animal before and after induction of MS-like symptoms, stored in appropriately marked Eppendorf tubes with anti-coagulant at 4° C. per standard procedures.

The first group of experimental mice are vaccinated with 100 μg each of mycobacteria that are suspended in standard complete Freund's adjuvant (CFA) at a concentration of 1 mg/ml, Accordingly, each mouse receives a subcutaneous injection of 0.1 ml of the suspended mycobacteria at the base of the tail. The second group of experimental mice are vaccinated with 2×10⁷ colony forming units of live Bacillus Calmette-Guerin (“BCG”) by intraperitoneal (“i.p.”) injection. The first control group of two mice are injected with 0.1 ml saline at the base of the tail; and the second control group of two mice are each injected i.p. with an equal volume of saline relative to the volume of BCG suspension that is injected i.p. into the second experimental group. All procedures are standard and are set forth in Yong et al., PLoS ONE 6(1):e16610 (2011) (wherein a BCG vaccine is shown to induce neuroprotection in a mouse model of Parkinson's Disease).

Twenty-four hours after injecting the two experimental sets and two control sets of mice, blood samples are collected from all mice of all four sets, and placed into appropriately marked tubes containing anti-coagulant. Analysis of the before and after injection sets of lymphocyte samples reveals newly induced lymphocytes that have CD3⁺/CD28⁺ markers from the mycobacteria- and BCG-injected mice, but no induced such lymphocytes in the control mice.

Application of BCG vaccination in human MS patients: A first lymphocyte sample is collected from the patient followed by a single intracutaneous dose of 0.1 ml of a 1 mg/ml solution of BCG (Berna Institute, Basel). Five to seven days after the administration of the BCG, a second lymphocyte sample is collected. The first lymphocyte sample and the second lymphocyte sample are subjected to clonotype analysis in accordance with Example 1.

Using the clonotype analysis, autologous lymphocytes are generated that express the identified receptors, in accordance with Example 2; and at least two billion daughter cells are generated.

One billion daughter cells are introduced into the patient in at least two successive weekly treatments of adoptive cell therapy. Primary outcome is measured by observation of number of gad-enhancing lesions in T1 in accordance with protocols well-known in the art.

Within six weeks of the first infusion of the adoptive cell therapy, the patient experiences reduction in symptoms.

### Example 7

This Example illustrates alternative protocols for treatment of Parkinson's disease (“PD”) using methods of the present invention.

First PD Treatment. A current treatment for advanced PD patients is deep brain stimulation (“DBS”), which is a surgical treatment for implanting a subcutaneous pacemaker that delivers an electric signal thus stimulating a location in the brain. Olanow et al., NEUROLOGY 55(12):S60-6 (2000). The invasive procedure, of necessity, also results in a specific tolerance induction event. It is also known that PD progression is associated with microglia activation, i.e., immune processes, resulting in inflammation contributed by the innate and adaptive immune systems. Panaro and Cianciulli, CURR. PHARM. DES. 18(2):200-8 (2012). Accordingly, the reversal of symptoms arising from the DBS must result in down-regulating the otherwise deleterious immune activity in advanced PD patients.

For treatment in accordance with the present invention, the advanced PD patient scheduled for the DBS procedure has a blood sample taken prior to the surgical procedure, and then again 7 days after the surgical procedure. Comparison of the pre- and post-STI event blood samples reveals the dominant TCR(s) that are then used to generate engineered autologous lymphocytes that express the said dominant TCRs. A therapeutic vaccine is then prepared with the autologous lymphocytes that express the dominant TCRs and increase down-regulation of the autoreactive activity associated with advancing PD.

Second PD Treatment. A second approach involving administration of Mycobacterium bovis bacillus-Calmette-Guerin (“BCG”) vaccine is supported in the literature by reported BCG vaccine-induced neuroprotection in a mouse model of PD, as stated by Yong et al, PLoS One 6(1):e16610 (2011). For this treatment: (1) a first lymphocyte sample is collected from the patient prior to vaccination, in accordance with Example 1; (2) a low dose (i.e., 0.1 ml of a BCG solution containing 1.6 to 3.2×10⁷ colony-forming units per ml of saline) of BCG vaccine is injected intradermally into the deltoid area of the patient, which injection causes a STI event; (3) seven days after the injection, a second lymphocyte sample is collected from the patient; (4) a clonotype analysis is conducted in accordance with the protocols of Example 1; (5) using methods set forth in Example 2, autologous lymphocytes that (a) express the receptors identified from the clonotype analysis are selected from a lymphocyte sample collected from the patient and/or (b) express the receptors identified from the clonotype analysis or those that are designed to enhance functionality based on the identified receptor and caused to be expressed in engineered autologous lymphocytes; and (6) the selected and/or engineered autologous lymphocytes are cultured in vitro to generate at least a billion daughter cells that are then used in an enhanced adoptive cell therapy as set forth in Example 2.

More particularly, the adoptive cell therapy for treating PD involves administering a dose of 10 million to 20 million cells per kg body weight by standard transfusion protocol. Administration of the same dose of cells is repeated weekly up to four weeks, after which the patient is observed for an additional four weeks. Patient's symptoms reduce significantly; but, if not, the four week regimen is repeated.

### Example 8

This Example illustrates one protocol for treatment of diabetes mellitus type I (T1D) using the method of the present invention.

Administration of a low dose of BCG was observed to cause a period of remission in some patients diagnosed with late-stage pre-diabetes. Faustman et al., PLoS ONE 7(8):e41756 (2012). Accordingly, a vaccine of BCG alone is usefully employed to create a STI event with regard to T1D.

For this treatment: (1) a first lymphocyte sample is collected from the patient prior to vaccination, in accordance with Example 1; (2) a low dose (i.e., 0.1 ml of a BCG solution containing 1.6 to 3.2×10⁷ colony-forming units per ml of saline) of BCG vaccine is injected intradermally into the deltoid area of the patient, which injection causes a STI event; (3) seven days after the injection, a second lymphocyte sample is collected from the patient; (4) a clonotype analysis is conducted in accordance with the protocols of Example 1; (5) using methods set forth in Example 2, autologous lymphocytes that (a) express the receptors identified from the clonotype analysis are selected from a lymphocyte sample collected from the patient and/or (b) express the receptors identified from the clonotype analysis or those that are designed to enhance functionality based on the identified receptors and caused to be expressed in engineered autologous lymphocytes; and (6) the selected and/or engineered autologous lymphocytes are cultured in vitro to generate at least a billion daughter cells that are then used in an enhanced adoptive cell therapy as set forth in Example 2.

More particularly, the adoptive cell therapy for treating T1D involves administering a dose of 10 million to 20 million cells per kg body weight by standard transfusion protocol. Administration of the same dose of cells is repeated weekly up to four weeks, after which the patient is observed for an additional four weeks. Patient's symptoms reduce significantly; but, if not, the four week regimen is repeated.

### Example 9

This Example illustrates one protocol for treatment of acute myocardial infarct or brain infarct using the method of the present invention.

Method for generating cells to be employed in an enhanced adoptive cell therapy for treating a patient afflicted with an acute myocardial infarct or a brain infarct (collectively, with respect to this example, “infarct” shall refer to either variety of infarct) involves the following:

First, clonotype analysis. Sequence DNA from (1) a first lymphocyte sample collected prior to and/or within one to two days after the infarct commences and (2) a second lymphocyte sample collected five to six days after the infarct commences. A second sampling of the second lymphocyte sample is preferably collected seven to eight days after the infarct commences, as a precaution in case the patient's immune response to the STI event is delayed. The lymphocyte samples are taken from peripheral blood. A clonotype analysis is conducted using the first lymphocyte sample and the second lymphocyte sample in accordance with the protocols set forth in Example 1 above. In so doing, the dominant lymphocyte receptor(s) associated with lymphocytes present in the patient upon response to the infarct are identified.

Second, creating cells for adoptive cell therapy. Using methods set forth in Example 2, autologous lymphocytes that (a) express the receptors identified from the clonotype analysis are selected from a lymphocyte sample collected from the patient and/or (b) express the receptors identified from the clonotype analysis or those that are designed to enhance functionality based on the identified receptor(s) and caused to be expressed in engineered autologous lymphocytes, which are created using standard protocols known in the art.

Third, conducting adoptive cell therapy. The selected and/or engineered autologous lymphocytes are cultured in vitro to generate at least a billion daughter cells that are then used in an enhanced adoptive cell therapy as set forth in Example 2. More particularly, the adoptive cell therapy for treating infarct involves administering a dose of about 20 million cells per kg body weight by standard transfusion protocol. Administration of the same dose of cells is repeated weekly up to four weeks, after which the patient is observed for an additional four weeks. It is our expectation that the patient's symptoms will have reduced significantly; but, if not, the four week regimen is repeated.

As it is the case that an expanded culture of the selected autologous cells that have the identified receptor(s) can be prepared faster than an expanded culture of engineered autologous cells that express the identified receptor(s) or enhanced versions thereof, the first treatment of adoptive cell therapy is commonly accomplished with the expanded culture of selected autologous cells.

### Example 10

This Example illustrates one protocol for treatment of atherosclerosis using the method of the present invention.

Inflammation is a hallmark throughout atherosclerotic lesion formation preceding acute myocardial infarct as well as at the time of plaque rupture. It is also known that there is a systemic decrease in T_{reg} cells associated with an increase in atherosclerotic plaques. Immunosuppression by, for example, mTOR inhibitors, causes an increased rate of conversion of T cells to T_{reg} cells, which is associated with reduction in the harms of atherosclerosis. Segundo et al., TRANSPLANTATION PROCEEDINGS 42:2871-2873 (2010). However, high degrees of immunosuppression, even with drug inhibitors of the mammalian target of rapamycin (“mTOR”), may be responsible for various adverse effects, including neoplasia and opportunistic infections. Nonetheless, use of mTOR inhibitors rapamycin (also known as sirolimus) and analogs thereof, such as everolimus, temsirolimus, and ridaforolimus, is an included protocol for treatment in the context of the present invention for treating atherosclerosis (particularly inasmuch rapamycin is associated with preventing coronary artery re-stenosis). We administer a rapamycin analog (a “rapalog”) to cause STI events in a protocol of the present invention for creating an enhanced adoptive cell therapy for atherosclerosis.

For this treatment: (1) a first lymphocyte sample is collected from the patient prior to administration of the mTOR inhibitor, in accordance with Example 1; (2) a low daily dose (i.e., an amount that results in low nM concentration in the patient's blood) of rapamycin is administered orally or intravenously to the patient—presence of the mTOR inhibitor causes a STI event; (3) at seven, 14, and 21 days after the first administration of the mTOR inhibitor, second lymphocyte samples are collected from the patient; (4) a clonotype analysis is conducted in accordance with the protocols of Example 1, taking note of the three different second lymphocyte samples; (5) using methods set forth in Example 2, autologous lymphocytes that (a) express the receptors identified from the clonotype analysis are selected from a lymphocyte sample collected from the patient and/or (b) express the receptors identified from the clonotype analysis or those that are designed to enhance functionality based on the identified receptor(s) and caused to be expressed in engineered autologous lymphocytes; and (6) the selected and/or engineered autologous lymphocytes are cultured in vitro to generate at least a billion daughter cells that are then used in an enhanced adoptive cell therapy as set forth in Example 2.

More particularly, the adoptive cell therapy for treating atherosclerosis involves administering a dose of 10 million to 20 million cells per kg body weight by standard transfusion protocol. Administration of the same dose of cells is repeated weekly up to four weeks, after which the patient is observed for an additional four weeks. The patient's symptoms reduce significantly; but, if not, the four week regimen is repeated.

### Example 11

This Example illustrates another protocol for treatment of atherosclerosis using an embodiment of the present invention.

Oral administration of Mycobacterium HSP60 has been shown to attenuate atherosclerotic lesions in experimental models as well as increase the number of T_{reg} cells. Puijvelde et al., ARTERIOSCLER. THROMB. VASC. BIOL. 27:2677-2683 (2007). We conclude that the oral administration of Mycobacterium HSP60 results in a STI event that is usefully employed in the context of the present invention. A treatment for addressing atherosclerosis, accordingly, includes the following steps:

First, a first lymphocyte sample is collected from the patient prior to administration of the Mycobacterium HSP60, in accordance with Example 1.

Second, a daily dose of Mycobacterium HSP60 is injected intravenously into the patient.

Third, at seven, 14, and 21 days after the first administration of the Mycobacterium HSP60, second lymphocyte samples are collected from the patient.

Fourth, a clonotype analysis is conducted in accordance with the protocols of Example 1, taking note of the three different second lymphocyte samples

Fifth, using methods set forth in Example 2, autologous lymphocytes that (a) express the receptors identified from the clonotype analysis are selected from a lymphocyte sample collected from the patient and/or (b) express the receptors identified from the clonotype analysis or those that are designed to enhance functionality based on the identified receptor(s) and caused to be expressed in engineered autologous lymphocytes.

Sixth, the selected and/or engineered autologous lymphocytes are cultured in vitro to generate at least a billion daughter cells that are then used in an enhanced adoptive cell therapy as set forth in Example 2.

More particularly, the adoptive cell therapy for treating atherosclerosis involves administering a dose of 10 million to 20 million cells per kg body weight by standard transfusion protocol. Administration of the same dose of cells is repeated weekly up to four weeks, after which the patient is observed for an additional four weeks. The patient's symptoms reduce significantly; but, if not, the four week regimen is repeated.

### Example 12

This Example illustrates one protocol for treatment of coronary artery disease (CAD) using the method of the present invention.

Patients afflicted with coronary artery disease (CAD) have been noted to have heightened sensitivity to influenza infection, which infection results in mortality and coronary symptoms at a significantly greater rate than does the general population. Accordingly, administration of influenza vaccine is not only strongly indicated for CAD patients, but is a STI event.

For this treatment: (1) a first lymphocyte sample is collected from the CAD patient prior to influenza vaccination, in accordance with Example 1; (2) a standard dose of influenza vaccine (Influvac, SolvayPharma) is injected intradermally into the deltoid area of the patient, which injection causes a STI event; (3) seven days after the injection, a second lymphocyte sample is collected from the patient; (4) a clonotype analysis is conducted in accordance with the protocols of Example 1; (5) using methods set forth in Example 2, autologous lymphocytes that (a) express the receptors identified from the clonotype analysis are selected from a lymphocyte sample collected from the patient and/or (b) express the receptors identified from the clonotype analysis or those that are designed to enhance functionality based on the identified receptors and caused to be expressed in engineered autologous lymphocytes; and (6) the selected and/or engineered autologous lymphocytes are cultured in vitro to generate at least a billion daughter cells that are then used in an enhanced adoptive cell therapy as set forth in Example 2.

More particularly, the adoptive cell therapy for treating CAD involves administering a dose of 10 million to 20 million cells per kg body weight by standard transfusion protocol. Administration of the same dose of cells is repeated weekly up to four weeks, after which the patient is observed for an additional four weeks. The patient's symptoms reduce significantly; but, if not, the four week regimen is repeated.

### Example 13

This Example illustrates one protocol for treatment of periodontitis using the method of the present invention.

Periodontal disease is an autoimmune disorder that is initiated by the deposition of bacterial plaque biofilm on the patient's teeth by Porphyromonas gingivalis bacteria that reside in virtually all human mouths. Persson, J. CLIN. PERIODONTOL. 32(Suppl. 6):39-53 (2005). Indeed, patients diagnosed with aggressive periodontitis have autoreactive antibodies to native collagen types I and II as well as other immune system maladies confirming this disease as one of autoimmunity. Hendler et al., J. DENT. RES. 89(12):1389-1394 (2010). The removal of the dental plaque, particularly as connected to the deep and aggressive cleaning that occurs with a periodontal visit involving tooth scaling and polishing, and planing and debridement, if necessary, to remove all mineralized plaque (tartar) found on the patient's teeth, is a STI event. Lymphocyte samples taken before and after the periodontal tooth cleaning is subjected to the clonotype analysis set forth in Example 1 to identify receptor(s) of lymphocyte cells that rise in concentration in response to the STI event. In particular:

First, a first lymphocyte sample is collected from the patient prior to commencing a periodontal tooth cleaning, in accordance with Example 1.

Second, the patient receives a periodontal tooth cleaning.

Third, at seven days after the periodontal cleaning, second lymphocyte sample is collected from the patient.

Fourth, a clonotype analysis is conducted in accordance with the protocols of Example 1.

Fifth, using methods set forth in Example 2, autologous lymphocytes that (a) express the receptors identified from the clonotype analysis are selected from a lymphocyte sample collected from the patient and/or (b) express the receptors identified from the clonotype analysis or those that are designed to enhance functionality based on the identified receptor(s) and caused to be expressed in engineered autologous lymphocytes.

Sixth, the selected and/or engineered autologous lymphocytes are cultured in vitro to generate at least a billion daughter cells that are then used in an enhanced adoptive cell therapy as set forth in Example 2.

More particularly, the adoptive cell therapy for treating periodontitis involves administering a dose of 10 million to 20 million cells per kg body weight by standard transfusion protocol. Administration of the same dose of cells is repeated weekly up to four weeks, after which the patient is observed for an additional four weeks. The patient's symptoms reduce significantly; but, if not, the four week regimen is repeated.

### Example 14

This Example illustrates one protocol for treatment of clinical depression using the method of the present invention.

Clinical depression has been shown to be accompanied by immunological effects on neuroplasticity. Eyre and Baune, PSYCHONEUROENDOCRINOLOGY 37:1397-1416 (2012c) (see, in particular, Table E, p. 1404, which tabulates cellular neuroimmunological effects on neuroplasticity during the course depression treatment); also, see Kovaru, NEURO ENDOCRINOL LETT. 30(4):421-8 (2009) and Liperoti, CURR PHARM DES. 15(36):4165-72 (2009). In depression, TNFα-mediated dysfunction of T cells has been observed, which results in loss of the neuroprotective function of T cells. Miller, Brain Behay. Immun. 24(1):1-8 (2010).

Accordingly, a therapeutic approach in line with the present invention is available, as follows:

First, a first lymphocyte sample is collected from the patient's peripheral blood prior to commencing treatment.

Second, the patient receives a dose of an anti-TNFα biological (e.g., infliximab, adalimumab, certolizumab pegol, golimumab), a circulating receptor fusion protein such as etanercept, or a small molecule TNF inhibitor (e.g., pentoxifylline or Bupropion), in accordance with Example 1 and using standard administration and dosing.

Third, at seven days after the drug administration, a second lymphocyte sample from the patient's peripheral blood is collected.

Fourth, a clonotype analysis is conducted in accordance with the protocols of Example 1.

Fifth, using methods set forth in Example 2, autologous lymphocytes that (a) express the receptors identified from the clonotype analysis are selected from a lymphocyte sample collected from the patient and/or (b) express the receptors identified from the clonotype analysis or those that are designed to enhance functionality based on the identified receptor(s) and caused to be expressed in engineered autologous lymphocytes.

Sixth, the selected and/or engineered autologous lymphocytes are cultured in vitro to generate at least a billion daughter cells that are then used in an enhanced adoptive cell therapy as set forth in Example 2.

More particularly, the adoptive cell therapy for treating clinical depression involves administering a dose of 10 million to 20 million cells per kg body weight by standard transfusion protocol. Administration of the same dose of cells is repeated weekly up to four weeks, after which the patient is observed for an additional four weeks. The patient's symptoms reduce significantly; but, if not, the four week regimen is repeated.

### Example 15

This Example illustrates one protocol for treatment of rheumatoid arthritis using the method of the present invention.

Rheumatoid arthritis is an autoimmune disease of the joints with acute exacerbation of inflammation of the synovia (i.e., lining of the joints). Over the years, local injections into the joints and systemic treatments have been tried with steroids and with immunosuppressant drugs, such as etanercept. None of the local injection-based treatments have shown persistently good results, although the initial specific self tolerance induced in these patients factors in our design of a protocol in the context of the present invention for treatment of rheumatoid arthritis.

The method of treatment includes harvesting lymphocytes from synovial fluid and serum as soon as there is clinical and laboratory evidence of improvement of the disease. These cells are isolated and analyzed as explained in example 1. The information obtained is utilized for specific enhanced adoptive therapy to enhance the treatment of the disease with a treatment that has fewer side effects than the immunosupressants. In particular, the method involves:

First, a first lymphocyte sample is collected from the patient's synovial fluid and/or peripheral blood prior to commencing treatment with etanercept, in accordance with Example 1.

Second, the patient receives a dose of etanercept.

Third, at seven days after the etanercept administration, a second lymphocyte sample from the patient's synovial fluid or blood is collected.

Fourth, a clonotype analysis is conducted in accordance with the protocols of Example 1.

Fifth, using methods set forth in Example 2, autologous lymphocytes that (a) express the receptors identified from the clonotype analysis are selected from a lymphocyte sample collected from the patient and/or (b) express the receptors identified from the clonotype analysis or those that are designed to enhance functionality based on the identified receptor(s) and caused to be expressed in engineered autologous lymphocytes.

Sixth, the selected and/or engineered autologous lymphocytes are cultured in vitro to generate at least a billion daughter cells that are then used in an enhanced adoptive cell therapy as set forth in Example 2.

More particularly, the adoptive cell therapy for treating rheumatoid arthritis involves administering a dose of 10 million to 20 million cells per kg body weight by standard transfusion protocol. Administration of the same dose of cells is repeated weekly up to four weeks, after which the patient is observed for an additional four weeks. The patient's symptoms reduce significantly; but, if not, the four week regimen is repeated.

### Example 16

This Example illustrates a second protocol for treatment of rheumatoid arthritis using the method of the present invention.

A rheumatoid arthritis patient is subjected to the following protocol:

1. A pre-STI event blood sample is drawn, in accordance with standard venipuncture methods.

2. Umbilical cord-derived mesenchymal stem cells (UC-MSCs) are administered to the patient, dosage of about 1×10⁷ cells per infusion, in accordance with Santos et al., J. TRANSL. MED. 11:18 (2013); also, see NCT01547091.

3. A post-STI event blood draw is taken two to three days after the infusion of UC-MSCs.

4. Lymphocytes respectively purified from the pre- and post-STI event blood draws are subjected to high throughput sequencing as described in Example 2.

5. Clonotypes determined to have increased significantly in abundance post-STI relative to the pre-STI data are then used to isolate additional cells of said clonotype from the patient and expanded in vitro.

6. Expanded cells are administered via transfusion to the patient under protocols set forth in Example 2.

7. Immunological End points: down regulation of pro-inflammatory cytokine and chemokine levels [TNFα, IL-6, monocyte chemoattractant protein-1, D-dimer (D-D), antithrombin-III (AT-III), thrombomodulin (TM)] and up regulation of the anti-inflammatory cytokine IL-10, as measured in sera Ponte et al., et al., STEM CELLS 25:1737-1745 (2007); Ringe et al., J. CELL BIOCHEM. 101:135-146 (2007); Soleymaninejadian et al., Am. J. Reprod. Immunol 67:1-8 (2012); De Miguel et al., Clin. Exp. Rheumatol., 30:S34-S38 (2012); Chapel et al., J. GENE MED. 5:1028-1038 (2003); Liu et al., ARTHRITIS RES. THER. 12:R210 (2010); Lin et al., CYTOTHERAPY 14:274-284 (2012); Weiss et al., STEM CELLS 24:781-792 0006); Cooper et al., STEM CELLS INT. 2011:905621 (2011).

### Example 17

This Example illustrates one protocol for treatment of brain infarct using the method of the present invention.

Stroke is the second leading cause of death and the leading cause of adult disability worldwide. After initial injury to the brain caused by the stroke event, the tissue tries to recover and heal itself using pro-inflammatory and anti-inflammatory factors in the area of infarction.

Only recently, it was discovered that phototherapeutic effects of low level infrared (808 nm) irradiation of brain neuronal cells protects the brain. It was reported recently that 710 nm wavelength visible light increases total lymphocyte counts in vivo, especially CD4⁺ T lymphocytes.

A stroke patient is subjected to the following protocol:

1. A “pre-STI event” blood sample is drawn within one day of stroke onset, and prior to irradiation with a light-emitting diode (LED), in accordance with standard venipuncture methods. (The medical chart of the patient is checked in case s/he had a blood draw within the recent past.)

2. A post-STI event blood draw is taken three to five days after stroke onset occurred and two to three days after the LED irradiation commences.

3. Lymphocytes respectively purified from the pre- and post-STI event blood draws are subjected to high throughput sequencing as described in Example 1.

4. Clonotypes determined to have increased significantly in abundance post-STI relative to the pre-STI data are then used to isolate additional cells of said clonotype from the patient and expanded in vitro.

5. Expanded cells are administered via transfusion to the patient under protocols set forth in Example 2.

Irradiation Procedure: A light-emitting diode (LED) light source is used with the following technical characteristics: peak wavelength 710 nm; radiant power 0.047 mW; irradiation area 1.13 cm²; power density 0.042 mW/cm²; energy density 1.796 J/cm², and luminous flux 0.054 mlm. The device is equipped with filters to block redundant light beyond the target wavelength range and is set towards the patient within 15 cm of the patient's cranium, with constant exposure to the LED light for 12 hours a day. The LED devices are sourced from Qray Inc. (Seongnam, Korea); the photometric features are measured by spectrometric instruments (e.g., CAS 140CT; Instrument Systems GmbH, Munich, Germany). The treatment is continued for up to 20 consecutive days with successive post-STI blood draws taken until a regulatory lymphocyte clonotype(s) are identified using the protocols of Example 1. Liesz et al., NAT MED 15:192-199 (2009); Offner et al., NEUROSCIENCE 158:1098-1111 (2009); Yan et al., J. NEUROIMMUNOL. 206:112-117 (2009); Una et al. Neuroscience 158:1174-1183 (2009); Vogelgesang et al., STROKE 39:237-241 (2008); Samoilova et al., PROC SPIE 3569:90-103 (1998); Samoilova et al., LASER TECHNOL. 9:40-41 (1999); Roberts et al., ANN. NY ACAD. SCI. 917:435-445 (2000).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treatment of a patient who suffers from an autoimmune disease, wherein the patient undergoes or experiences a specific tolerance induction (“STI”) event, the method comprising the steps of:
(a) optionally collecting a first lymphocyte-containing sample from the patient prior to the STI event (“pre-STI event sample”);
(b) detecting an STI event or performing a procedure that correlates in time to an STI event;
(c) collecting a second lymphocyte-containing sample from the patient after the STI event (“post-STI event sample”);
(d) preparing lymphocytes from the pre-STI event sample or the post-STI event sample;
(e) preparing and sequencing DNA or cDNA derived from the prepared lymphocytes;
(f) identifying sequences of prevalent T cell receptors (“TCRs”) or B cell receptors (“BCRs”) (collectively, “prevalent receptor sequences”) derived from the post-STI event sample;
(g) selecting a regulatory lymphocyte that carries at least one of the prevalent receptor sequences or a sequence that is at least 85% related to one of the prevalent receptor sequences, which selected regulatory lymphocyte (i) expresses one or more prevalent receptor sequences or (ii) is generated from an autologous or allogeneic naïve lymphocyte, which naïve lymphocyte is engineered and induced to become a regulatory lymphocyte that expresses at least one prevalent receptor sequence;
(h) culturing the selected regulatory lymphocyte, thereby generating daughter cells of said regulatory lymphocyte; and
(i) administering said daughter cells to said patient.

2. The method of claim 1, wherein if the first lymphocyte-containing sample was collected from the patient, then step (f) hereof further comprises identifying one or more prevalent receptor sequences derived from the post-STI event sample relative to TCRs or BCRs derived from the pre-STI event sample.

3. The method of claim 1, wherein the autoimmune disease is selected from the group consisting of Addison's disease, alopecia, Alzheimer's disease, amyotrophic lateral sclerosis (“ALS”), aplastic anemia, autoimmune brain infarction, autoimmune coronary artery disease, autoimmune myocardial infarction, autoimmune periodontal disease, brain trauma, celiac disease, Crohn's disease, diabetes mellitus type I, glaucoma, glutamate toxicity, idiopathic thrombocytopenic purpura, interstitial cystitis, multiple sclerosis, Parkinson's disease, psoriasis, rheumatoid arthritis, spinal cord trauma, systemic lupus erythematosus, and systemic sclerosis.

4. The method of claim 1, wherein the STI event is caused by or correlates proximately in time to subjecting the patient to a protocol selected from the group consisting of (a) injecting weakened Mycobacterium bovis or Mycobacterium tuberculosis, (b) applying ultraviolet A irradiation, (c) Goeckerman therapy, (d) injecting an amyloid beta vaccine, (e) injecting an influenza vaccine, (f) prophylaxis dental treatment, and (g) surgery.

5. The method of claim 1, wherein the STI event is a flare-up of an autoimmune disease, a myocardial infarct, or a brain infarct.

6. The method of claim 1, wherein the daughter cells are administered in combination with a cytokine.

7. The method of claim 6, wherein the cytokine is IL-10 or TGF-β.

8. The method of claim 1, wherein step (a) is not optional.

9. The method of claim 8, further comprising the step of identifying clonotypes from the post-STI event sample that are not identified among clonotypes from the pre-STI event sample or that have an expansion frequency of 0.5% or greater relative to the clonotypes from the pre-STI event sample.

10. The method of claim 9, wherein identifying clonotypes comprises spectratype analysis or sequencing DNA or cDNA.

11. The method of claim 1, further comprising the steps of:
(a) identifying one or more V or J segments among the prevalent receptor sequences; and
(b) selecting a regulatory lymphocyte bearing the identified V or J segment(s).

12. The method of claim 1, further comprising the steps of:
(a) enhancing the efficacy of the selected regulatory lymphocyte in vitro.

13. A method for identifying regulatory lymphocyte clonotypes that have expanded in a patient following a specific tolerance induction (“STI”) event or a procedure that correlates in time to an STI event, comprising the steps of:
(a) optionally collecting a first lymphocyte-containing sample from the patient, wherein the first lymphocyte-containing sample is collected prior to the STI event;
(b) recording the STI event or performing the procedure on the patient;
(c) collecting a second lymphocyte-containing sample from the patient, wherein the second lymphocyte-containing sample is collected subsequent to the STI event;
(d) spectratyping lymphocytes from the first lymphocyte-containing sample, if said first lymphocyte-containing sample was collected, and from the second lymphocyte-containing sample; and
(e) identifying regulatory lymphocyte clonotypes that have expanded following the STI event.

14. The method of claim 13, further comprising the steps of:
(a) purifying lymphocytes from the first lymphocyte-containing sample; and
(b) purifying lymphocytes from the second lymphocyte-containing sample.

15. A therapeutic composition for treating a patient afflicted with an autoimmune disease, which composition includes ex vivo cultured regulatory lymphocytes that are substantially enriched for those that express one receptor sequence.

16. The therapeutic composition of claim 15, wherein the receptor sequence is identified from or based upon analysis of a lymphocyte-containing sample that was purified after removal from the patient.

17. The therapeutic composition of claim 15, wherein the ex vivo cultured regulatory lymphocytes express two different receptor sequences.

18. The therapeutic composition of claim 15, further comprising a cytokine.

19. The therapeutic composition of claim 15, wherein the ex vivo cultured regulatory lymphocytes, subsequent to administration into the patient, specifically suppress one or more immune system functions involved in the patient's autoimmune disease.

20. The therapeutic composition of claim 15, wherein the ex vivo cultured regulatory lymphocytes are derived from an autologous lymphocyte isolated from the patient or a lymphocyte that is allogeneic with respect to the patient.
